(19) United States
(12) Patent Application Publication
(10) Pub. No.: US 2021/0236720 A1
(43) Pub. Date: Aug. 5, 2021

(54) CHEMICAL SOLUTION PUMP AND CHEMICAL SOLUTION ADMINISTRATION DEVICE

(71) Applicant: Seiko Instruments Inc., Chiba-shi (JP)

(72) Inventors: Tetsuya NAGATA, Chiba-shi (JP); Akehiko SATO, Chiba-shi (JP); Yoichi ENDO, Chiba-shi (JP)

(21) Appl. No.: 17/155,873

(22) Filed: Jan. 22, 2021

(30) Foreign Application Priority Data
Jan. 31, 2020 (JP) 2020-015399

(51) Int. Cl. A61M 5/145

(52) U.S. Cl. CPC A61M 5/1456

(57) ABSTRACT

The present invention relates to a chemical solution pump including a holding member that holds a syringe discharging a chemical solution in association with a movement of a plunger, a drive unit that presses the plunger with a predetermined driving force to move the plunger in a first direction toward an inside of the syringe, and an adjustment mechanism that adjusts the movement of the plunger so that the plunger moves with the driving force. The adjustment mechanism applies an auxiliary force to the plunger in a direction the same as the first direction, or applies a reaction force to the plunger in a second direction opposite to the first direction, in accordance with a difference between a resistance force generated by the movement of the plunger inside the syringe and the driving force.

### RELATED APPLICATIONS

This application claims priority to Japanese Patent Application No. 2020-015399, filed on Jan. 31, 2020, the entire content of which is incorporated herein by reference.

### BACKGROUND OF THE INVENTION

### 1. Field of the Invention

The present invention relates to a chemical solution pump and a chemical solution administration device.

### 2. Description of the Related Art

In the related art, a chemical solution pump that feeds a chemical solution has been used in various fields. For example, a chemical solution pump is used when the chemical solution is injected in physical and chemical fields and a medical field, when the chemical solution such as acid and alkali is injected in a water treatment field, or when the chemical solution such as a nutritional supplement is injected in a livestock field.

As the chemical solution pump of this type, for example, as disclosed in Japanese Unexamined Patent Application, First Publication No. 2011-250867, a chemical solution pump is known which discharges a chemical solution such as insulin and administers the chemical solution into a user's body.

The chemical solution pump includes a holding body that holds a syringe filled with the chemical solution, a pressing member provided in the holding body to be movable and pressing a plunger of a syringe, a spiral spring that biases the pressing member together with the plunger, and locking means for restricting a movement of the pressing member.

According to the chemical solution pump configured in this way, when movement restriction of the pressing member by the locking means is released, the spiral spring elastically deforms to wind up a portion connected to the pressing member due to the spiral spring's own elastic restoring force. In this manner, the pressing member can be moved to press the plunger. As a result, the chemical solution can be discharged from the inside of the syringe with a constant discharge amount (discharge speed), and can be administered into the user's body.

### SUMMARY OF THE INVENTION

In the above-described chemical solution pump in the related art, the discharge amount of the chemical solution is keeping constant by using power of the spiral spring to press the pressing member.

However, when the plunger is moved inside the syringe, the plunger actually receives a resistance force, since the plunger is affected by sliding resistance of a sealing member such as an O-ring or a gasket disposed inside the syringe, or viscosity of the chemical solution. Moreover, the resistance force is not always constant, and may fluidly be changed during the movement of the plunger in some cases. Therefore, it is difficult to press the plunger with a predetermined driving force (thrust), and a movement amount of the plunger per unit time tends to fluctuate. Therefore, in the above-described chemical solution pump in the related art, it is difficult to maintain the constant discharge amount of the chemical solution, and moreover, there is a problem in that discharge accuracy is degraded.

The present invention is made in view of the above-described circumstances, and an object thereof is to provide a chemical solution pump and a chemical solution administration device which are capable of accurately discharging a chemical solution with a constant discharge amount.

(1) According to a first aspect of the present invention, there is provided a chemical solution pump including a holding member that holds a syringe filled with a chemical solution and discharging the chemical solution in association with a movement of a plunger disposed to be slidably movable inside the syringe, a drive unit that presses the plunger with a predetermined driving force to move the plunger in a first direction toward an inside of the syringe, and an adjustment mechanism that adjusts the movement of the plunger so that the plunger moves with the driving force. The adjustment mechanism applies an auxiliary force to the plunger in a direction the same as the first direction, or applies a reaction force to the plunger in a second direction opposite to the first direction, in accordance with a difference between a resistance force generated by the movement of the plunger inside the syringe and the driving force.

In this case, the plunger is pressed by using the driving force of the drive unit. Therefore, the plunger can be moved to be pushed into the syringe, and the chemical solution inside the syringe can be discharged outward. In particular, when the plunger is pressed by using the driving force, the adjustment mechanism applies the auxiliary force to the plunger in the first direction toward the inside of the syringe, or applies the reaction force to the plunger in the second direction opposite to the first direction, in accordance with the difference between the resistance force generated by the movement of the plunger inside the syringe and the driving force.

For example, when the resistance force (for example, sliding resistance generated between the syringe and the plunger or viscous resistance of the chemical solution) generated by the movement of the plunger is great, a movement speed of the plunger becomes slower, and a movement amount per unit time of the plunger decreases. In this case, the adjustment mechanism increases the speed of the plunger by applying the auxiliary force (that is, positive thrust) to the plunger in the first direction toward the inside of the syringe. On the other hand, when the resistance force is weak, the movement speed of the plunger becomes faster, and the movement amount per unit time of the plunger increases. In this case, the adjustment mechanism can prevent the movement speed of the plunger from becoming faster by applying the reaction force (that is, negative thrust) to the plunger in the second direction opposite to the first direction.

In this way, the adjustment mechanism applies the auxiliary force or the reaction force to the plunger, in accordance with the difference between the resistance force generated by the movement of the plunger inside the syringe and the driving force. Therefore, the movement speed can be corrected to offset influence of the resistance force. Therefore, the plunger can be moved by the driving force applied from the drive unit. Therefore, it is possible to feed and move (feed-move) the plunger with a constant movement amount, and the chemical solution can be accurately discharged with a constant discharge amount (constant amount). Therefore, it is possible to provide the chemical solution pump having excellent discharge accuracy. The chemical solution pump includes the drive unit. Accordingly, for example, even when the auxiliary force itself applied by the adjustment mechanism is weaker than the resistance force, it is possible to feed and move (feed-move) the plunger with a constant movement amount.

(2) The adjustment mechanism may include a movable body disposed to be movable along an axis in conjunction with the plunger, a feed mechanism that moves the movable body in the first direction at a predetermined speed, and applies the auxiliary force to the plunger via the movable body, and a braking unit that applies the reaction force to the plunger in the second direction via the movable body.

In this case, the adjustment mechanism includes the feed mechanism and the braking unit. Accordingly, it is possible to properly apply the auxiliary force or the reaction force to the plunger in accordance with the difference between the resistance force and the driving force. The plunger can be reliably moved by the driving force applied from the drive unit.

(3) The movable body may have a feed screw having a male screw portion formed on an outer peripheral surface, and disposed in a state where rotation around the axis is restricted. The feed mechanism may include a nut member that has a female screw portion screwed to the male screw portion, and is screwed to the feed screw, a drive source that generates power for rotating the nut member, a train wheel mechanism that transmits the power from the drive source to the nut member, and a speed control mechanism that controls a speed of the train wheel mechanism. The braking unit may use at least a meshing force in the train wheel mechanism and a meshing force of the male screw portion with respect to the female screw portion, as the reaction force.

In this case, the train wheel mechanism transmits the power generated by the drive source to the nut member so that the nut member can be rotated around the axis. The male screw portion of the feed screw is screwed to the female screw portion of the nut member in a state where the rotation around the axis is restricted. Accordingly, the feed screw does not rotate together with the rotation of the nut member. Therefore, in association with the rotation of the nut member, the feed screw can be moved along the axis in the first direction. In this case, the speed of the train wheel mechanism is controlled by the speed control mechanism. Accordingly, the nut member can be rotated at a predetermined rotation speed. Therefore, in association with the rotation of the nut member, the feed screw can be moved at a predetermined speed in the first direction. In this manner, the auxiliary force can be properly applied to the plunger via the feed screw.

When the resistance force generated by the movement of the plunger is weak, the movement speed of the plunger becomes faster. Consequently, the feed screw provided in conjunction with the plunger is in a pulled state in the first direction. In this case, at least the meshing force in the train wheel mechanism and the meshing force between the male screw portion of the feed screw and the male screw portion of the nut member can be used as the reaction force. Therefore, it is possible to prevent the movement speed of the plunger from becoming faster.

(4) The drive source may have a mainspring that generates the power by an unwinding operation.

In this case, as in a case of a mechanical timepiece, the unwinding operation of the mainspring is used to generate the power for rotating the nut member. Accordingly, electric power of a battery is not needed to discharge the chemical solution. Therefore, it is possible to provide the chemical solution pump which achieves low cost and improved safety.

(5) The feed mechanism may include a switching mechanism that switches between stopping and starting power transmission from the drive source to the nut member. The feed mechanism may stop the movement of the feed screw and the plunger by stopping the power transmission to the nut member, and may move the plunger, based on the driving force while causing the adjustment mechanism to adjust the movement of the plunger by starting the power transmission to the nut member.

In this case, the switching mechanism can be used to switch between stopping and starting the power transmission from the drive source to the nut member. Accordingly, for example, the nut member can be rotated at any desired timing, or a time for rotating the nut member can be adjusted. In particular, the rotation of the nut member can be stopped to stop the movement of the feed screw itself provided in conjunction with the plunger. Accordingly, the movement of the plunger into the syringe can be stopped.

Therefore, it is possible to adjust a discharge timing or a discharge time of the chemical solution from the syringe. Accordingly, it is possible to provide the chemical solution pump which achieves convenient use and excellent discharge performance.

(6) The speed control mechanism may include an impeller that meshes with the train wheel mechanism, rotated by the power associated with the unwinding operation of the mainspring. The impeller may generate resistance corresponding to a rotation speed of the train wheel mechanism to control the speed of the train wheel mechanism. The switching mechanism may include a switching mainspring that generates switching power by an unwinding operation, and a movable member that moves between a separation position separated from the impeller and a stop position in contact with the impeller to stop rotation of the impeller, based on the switching power.

In this case, the speed of the train wheel mechanism can be controlled by using the resistance of the impeller. Accordingly, for example, unlike a speed control mechanism using a balance with hairspring in a mechanical timepiece, sound is less likely to be generated. Therefore, while maintaining quietness, the speed can be controlled.

Furthermore, the switching mechanism uses the switching power associated with the unwinding operation of the switching mainspring so that the movable member is moved between the separation position and the stop position. In particular, the movable member is moved to the stop position. In this manner, the rotation of the impeller meshing with the train wheel mechanism can be stopped. Accordingly, the rotation of the train wheel mechanism itself can be stopped to stop the rotation of the nut member. In this manner, it is possible to stop the movement of the feed screw provided in conjunction with the plunger and the movement of the plunger. In particular, as in a case of a mechanical timepiece, the switching mechanism can be configured to use the unwinding operation of the switching mainspring. Accordingly, electric power of a battery is not needed. Therefore, it is possible to control an operation timing of the constant amount feed of the plunger without using the electric power.

(7) The drive unit may include a spring member that generates the driving force by using an elastic restoring force.

In this case, the drive unit can have a simple configuration using various spring members such as a spiral spring, a leaf spring, a coil spring, a torsion spring, a disc spring, and a volute spring. Therefore, the drive unit easily achieves low cost and a simplified configuration.

(8) According to a second aspect of the present invention, there is provided a chemical solution administration device including the chemical solution pump, a main body case internally accommodating the chemical solution pump, and mountable on a living body surface, and an indwelling needle capable of indwelling the living body surface in a state where a living body is punctured, and into which the chemical solution discharged from the syringe is introduced.

In this case, the chemical solution discharged from the syringe by the chemical solution pump can be administered to the living body through the indwelling needle. In particular, the chemical solution pump is used so that the chemical solution can be accurately discharged from the syringe with a constant discharge amount (constant amount). Accordingly, for example, a determined amount of the chemical solution can be administered accurately and periodically. Therefore, for example, the chemical solution administration device can be suitably used as an insulin administration device for administering insulin into the body.

According to the aspect of the present invention, it is possible to provide the chemical solution pump and the chemical solution administration device which are capable of accurately discharging the chemical solution with the constant discharge amount.

### BRIEF DESCRIPTION OF THE DRAWINGS

FIG. 1 is a view illustrating a first embodiment of a chemical solution pump and a chemical solution administration device according to the aspect of the present invention, and is a perspective view illustrating a configuration of the whole chemical solution administration device.

FIG. 2 is a block diagram illustrating a simplified configuration of the chemical solution pump illustrated in FIG. 1.

FIG. 3 is a perspective view of the chemical solution pump illustrated in FIG. 1.

FIG. 4 is a top view of the chemical solution pump illustrated in FIG. 3.

FIG. 5 is a perspective view illustrating a state where an inner case and a spiral spring are detached from a state illustrated in FIG. 3.

FIG. 6 is a perspective view illustrating a state where a syringe is detached from the state illustrated in FIG. 3.

FIG. 7 is a perspective view of the spiral spring and the inner case which are illustrated in FIG. 6.

FIG. 8 is a perspective view illustrating a state where a feed wheel and a periphery of a nut member are detached from the state illustrated in FIG. 3.

FIG. 9 is a perspective view illustrating a state where a first cover and a second cover are detached from the state illustrated in FIG. 3.

FIG. 10 is a perspective view illustrating a state where a second guide member is detached from a state illustrated in FIG. 9.

FIG. 11 is a perspective view when the state illustrated in FIG. 3 is viewed from different viewpoints.

FIG. 12 is a view for describing an operation of the chemical solution pump according to the first embodiment, and is a view illustrating a relationship between a movement amount of a plunger and an elapsed time.

FIG. 13 is a top view illustrating a second embodiment of a chemical solution pump according to the aspect of the present invention.

FIG. 14 is a view illustrating a configuration of a switching mechanism illustrated in FIG. 13.

FIG. 15 is a view illustrating a relationship between a balance wheel and an oscillator plate which are illustrated in FIG. 14.

FIG. 16 is a view illustrating a state where the oscillator plate is moved to a stop position from a state illustrated in FIG. 14.

FIG. 17 is a view illustrating a state where the oscillator plate is moved to the stop position from a state illustrated in FIG. 15.

FIG. 18 is a view illustrating a state where the oscillator plate is moved to a start position from the state illustrated in FIG. 14.

FIG. 19 is a view for describing an operation of the chemical solution pump according to the second embodiment, and is a view illustrating a relationship between a movement amount of a plunger and an elapsed time when the plunger is operated to continuously administer a chemical solution after a lapse of a predetermined time.

FIG. 20 is a view illustrating a relationship between the movement amount of the plunger and the elapsed time when the plunger is operated to intermittently administer the chemical solution after a lapse of a predetermined time.

FIG. 21 is a view illustrating a relationship between a pulse voltage for a shape memory alloy wire and discharge.

FIG. 22 is a view illustrating a relationship between the movement amount of the plunger and the elapsed time when the plunger is operated to irregularly administer the chemical solution after a lapse of a predetermined time.

FIG. 23 is a perspective view illustrating a third embodiment of a chemical solution pump according to the aspect of the present invention.

FIG. 24 is a top view illustrating a fourth embodiment of a chemical solution pump according to the aspect of the present invention.

FIG. 25 is a perspective view of the chemical solution pump illustrated in FIG. 24.

FIG. 26 is a perspective view of the chemical solution pump illustrated in FIG. 24.

FIG. 27 is a perspective view of the chemical solution pump illustrated in FIG. 24.

FIG. 28 is a perspective view of the chemical solution pump illustrated in FIG. 24.

FIG. 29 is a view illustrating a modification example of an adjustment mechanism according to the aspect of the present invention.

FIG. 30 is a view illustrating another modification example of the adjustment mechanism according to the aspect of the present invention.

FIG. 31 is a view illustrating still another modification example of the adjustment mechanism according to the aspect of the present invention.

FIG. 32 is a view illustrating yet another modification example of the adjustment mechanism according to the aspect of the present invention.

### DETAILED DESCRIPTION OF THE PREFERRED EMBODIMENTS

### First Embodiment

Hereinafter, a first embodiment of a chemical solution pump and a chemical solution administration device according to the aspect of the present invention will be described with reference to the drawings.

### Chemical Solution Administration Device

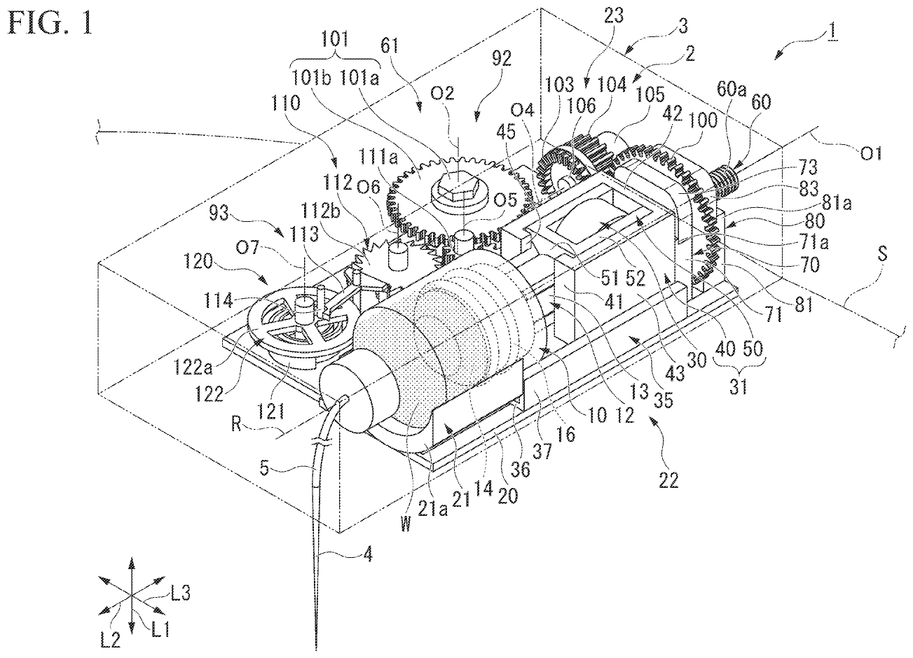

As illustrated in FIG. 1, a chemical solution administration device 1 of the present embodiment includes a chemical solution pump 2 that discharges a chemical solution W from a syringe 10 filled with the chemical solution W, and a main body case 3 internally accommodating the chemical solution pump 2, and mountable on a user's body surface (living body surface according to the present invention) S.

The chemical solution W is not particularly limited, and for example, insulin may be used. In this case, the chemical solution administration device 1 functions as an insulin administration device, and the chemical solution pump 2 functions as an insulin pump.

For example, the main body case 3 is configured so that a case body and a lid member are combined with each other, and can be mounted on a user's predetermined mounting location (for example, around an abdomen). In the present embodiment, the main body case 3 is formed in a rectangular parallelepiped shape having a box shape in order to simplify the illustration. However, a shape of the main body case 3 is not limited to this case. For example, the main body case 3 may be formed in a circular shape, an elliptical shape, or a polygonal shape in a plan view.

A mounting method for mounting the main body case 3 on the user's body surface S is not particularly limited, and a known method may be adopted. For example, the main body case 3 may be mounted on the body surface S by using an adhesive tape. Alternatively, a mounting member (not illustrated) such as a clip or a mounting belt may be combined with the main body case 3 so that the main body case 3 is mounted on the body surface S via the mounting member.

The main body case 3 has an indwelling needle 4 that can protrude into a body by using an operation member (not illustrated) which can perform a pushing operation.

For example, the indwelling needle 4 is a plastic-made cannula type, can puncture into the body together with an inner needle (not illustrated), and can indwell the body surface S by pulling out the inner needle. In this manner, the indwelling needle 4 can indwell the body surface S in a state of puncturing into the body while the main body case 3 is mounted on the body surface S.

The indwelling needle 4 is connected to the inside of the syringe 10 through a flexible tube 5, for example. Therefore, the chemical solution W is discharged from the syringe 10 by using the chemical solution pump 2. In this manner, the discharged chemical solution W can be introduced into the indwelling needle 4, and the chemical solution W can be administered to a user through the indwelling needle 4.

### Chemical Solution Pump

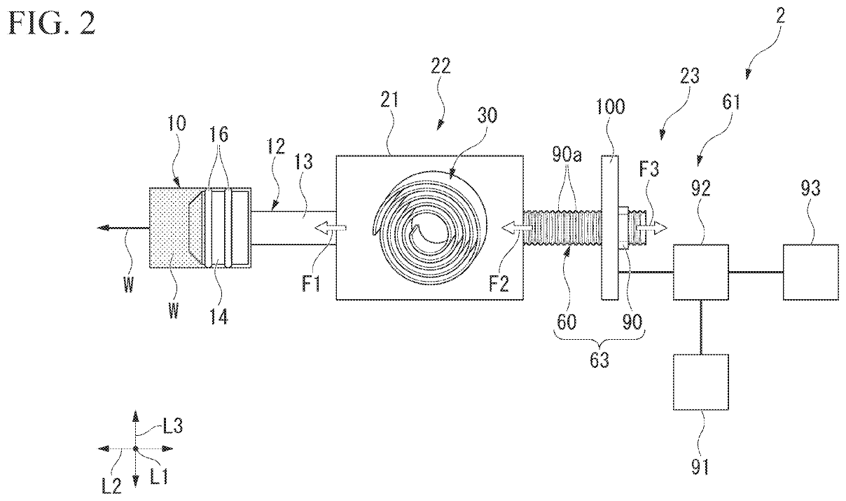

As illustrated in FIGS. 1 to 4, the chemical solution pump 2 includes a flat plate-shaped base plate 20 fixed inside the main body case 3. Each component forming the chemical solution pump 2 is mounted on the base plate 20. FIG. 2 is a simple block diagram illustrating a simplified configuration of the chemical solution pump 2.

In the present embodiment, a thickness direction of the base plate 20 will be referred to as an upward-downward direction L1, a direction separated from the body surface S will be referred to as upward, and a direction opposite thereto will be referred to as downward. Furthermore, in a plane of the base plate 20, in directions orthogonal to each other, one direction will be referred to as a forward-rearward direction L2, and the other direction will be referred to as a rightward-leftward direction L3.

### Syringe

First, the syringe 10 set in the chemical solution pump 2 will be briefly described.

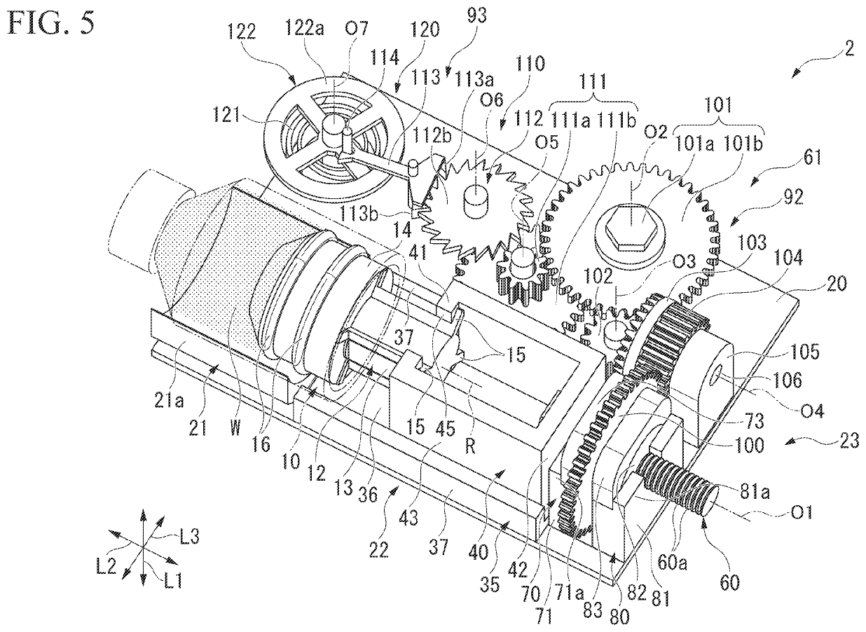

As illustrated in FIGS. 1 and 5, the syringe 10 is a so-called chemical solution container, and includes a plunger 12 disposed to be slidable inside the syringe 10. The syringe 10 is held by a holding member 21 (to be described later) so that a syringe axis R is parallel to the forward-rearward direction L2.

The syringe 10 extends along the forward-rearward direction L2, and is formed in a cylindrical shape around the syringe axis R so that the syringe 10 can be filled with the chemical solution W.

In the forward-rearward directions L2, a direction in which the plunger 12 is pushed into the syringe 10 will be referred to as a forward direction (first direction according to the present invention), and a direction opposite thereto will be referred to as a rearward direction (second direction according to the present invention). Furthermore, when viewed in the forward-rearward direction L2, a direction intersecting with the syringe axis R will be referred to as a radial direction, and a direction turning around the syringe axis R will be referred to as a circumferential direction.

An opening portion is formed on a rear end portion side of the syringe 10. Therefore, the syringe 10 is open to a rear side when set in the chemical solution pump 2. A tube 5 connected to the indwelling needle 4 can be connected to a front end portion side of the syringe 10. In this manner, the inside of the syringe 10 and the inside of the indwelling needle 4 can communicate with each other through the tube 5, and the chemical solution W can be supplied to the indwelling needle 4 from the inside of the syringe 10.

The plunger 12 is inserted into the syringe 10 from behind through the opening portion of the syringe 10. The plunger 12 includes a plunger shaft 13 extending along the forward-rearward direction L2, a columnar gasket portion 14 integrally formed in a front end portion of the plunger shaft 13, and a connection piece 15 integrally formed in a rear end portion of the plunger shaft 13.

The gasket portion 14 is slidable forward and rearward along the syringe axis R inside the syringe 10. A sealing member 16 such as an O-ring is fixed to an outer peripheral surface of the gasket portion 14. In this manner, the gasket portion 14 and the syringe 10 are sealed in a tight (liquid-tight and airtight) manner.

A plurality of the connection pieces 15 are formed to protrude outward in the radial direction from a rear end portion of the plunger shaft 13, and are formed at an interval in the circumferential direction. In the illustrated example, four connection pieces 15 are formed at an equal interval in the circumferential direction to be disposed radially around the syringe axis R.

However, a shape or the number of the connection pieces 15 is not limited to this case. For example, the annular connection piece 15 may be formed so that an entire periphery protrudes outward in the radial direction from the rear end portion of the plunger shaft 13.

For example, the syringe 10 configured as described above can be filled with the chemical solution W by transferring or suctioning the chemical solution W from a vial (also referred to as an ampoule) previously filled with the chemical solution W.

### Chemical Solution Pump

As illustrated in FIGS. 1 to 4, the chemical solution pump 2 includes the holding member 21 that holds the syringe 10, the drive unit 22 that presses the plunger 12 with a predetermined driving force F1 to move the plunger 12 forward into the syringe 10, the adjustment mechanism 23 that adjusts the movement of the plunger 12 so that the plunger 12 moves with the driving force F1.

The holding member 21, the drive unit 22, and the adjustment mechanism 23 are mounted on the upper surface side of the base plate 20 as described above.

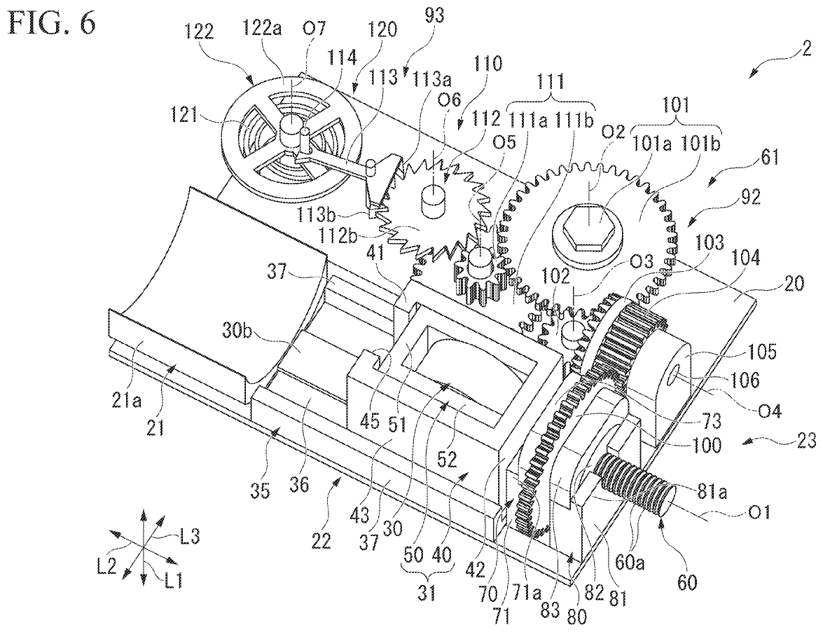

As illustrated in FIG. 6, the holding member 21 includes a holding base 21a disposed close to a front edge portion in the base plate 20, and a holding tool (not illustrated) combined with the holding base 21a.

The holding base 21a is formed to have a square or rectangular outer shape in a top view, and a size thereof corresponds to a diameter and a length of the syringe 10. An upper surface of the holding base 21a is an arc surface curved in the rightward-leftward direction L3 with a curvature corresponding to an outer diameter of the syringe 10. In this manner, the syringe 10 can be placed on the upper surface of the holding base 21a in a state of being positioned in the rightward-leftward direction L3.

Since the holding tool is combined with the holding base 21a, it is possible to hold the syringe 10 placed on the holding base 21a. In this manner, the syringe 10 can be stably and reliably held by using the holding member 21.

As illustrated in FIGS. 1 to 4, the drive unit 22 includes a spiral spring (spring member according to the present invention) 30 that generates the driving force F1 by using an elastic restoring force, a movable case 31 internally accommodating the spiral spring 30 and disposed to be movable forward, and a guide plate 35 that guides the movable case 31 to be movable.

As illustrated in FIG. 6, the guide plate 35 is disposed on a rear side of the holding member 21 and is integrally combined with the base plate 20.

The guide plate 35 includes a flat plate-shaped plate body 36 disposed to overlap the base plate 20, and a pair of guide rails 37 formed to protrude upward from the plate body 36 and to extend along the forward-rearward direction L2.

For example, the plate body 36 is formed in a rectangular shape in a plan view in which a length along the forward-rearward direction L2 is longer than a length along the rightward-leftward direction L3. A front end portion of the plate body 36 is in contact with or close to a rear end portion of the holding base 21a. An upper surface of the plate body 36 is formed to be smooth and is a sliding surface having low frictional resistance, for example.

Each of the pair of guide rails 37 is formed on a side edge portion located on both sides of the plate body 36 in the rightward-leftward direction L3, and is formed over an entire length of the plate body 36. Therefore, the pair of guide rails 37 is disposed parallel to each other in a state of facing each other in the rightward-leftward direction L3. In the pair of guide rails 37, a facing surface (inner surface) on which the guide rails 37 face each other is formed to be smooth, and is a sliding surface having low frictional resistance, for example.

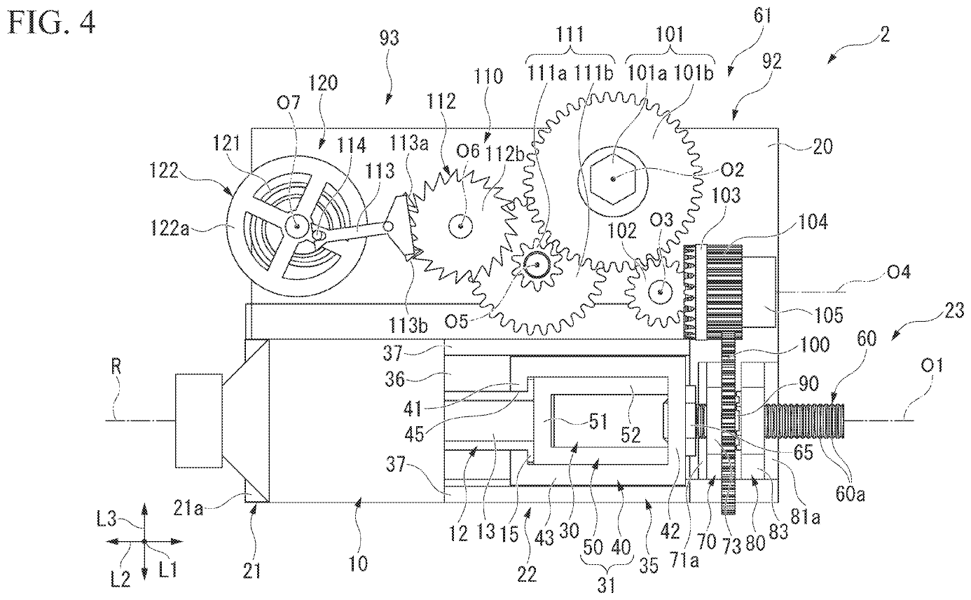

As illustrated in FIGS. 4 to 7, the movable case 31 includes an outer case 40 and an inner case 50 accommodated inside the outer case 40, and is placed on the upper surface of the plate body 36 in the guide plate 35.

The outer case 40 includes an outer front wall 41 and an outer rear wall 42 which are disposed to face each other in the forward-rearward direction L2, and a pair of outer side walls 43 disposed to face each other in the rightward-leftward direction L3, and is formed in a frame shape which is open upward and downward. In the illustrated example, an outer shape of the outer case 40 is a rectangular shape in a plan view in which the length along the forward-rearward direction L2 is longer than the length along the rightward-leftward direction L3.

The pair of outer side walls 43 is disposed inside the pair of guide rails 37, and is in contact with or close to the guide rails 37. In this manner, the whole movable case 31 can be moved on the upper surface of the plate body 36 in the forward-rearward direction L2 with less rattling while being guided by the pair of guide rails 37. Therefore, the whole movable case 31 can be moved in the forward-rearward direction L2 in an excellent straight manner.

The outer front wall 41 has an insertion hole 45 that penetrates the outer front wall 41 in the forward-rearward direction L2 and is open upward and downward. The plunger shaft 13 enters the inside of the outer case 40 from the rear side through the insertion hole 45. In this manner, the connection piece 15 formed in a rear end portion of the plunger shaft 13 is disposed inside the outer case 40. The connection piece 15 is in contact with the outer front wall 41 from the rear side.

The inner case 50 includes inner front walls 51 facing each other in the forward-rearward direction L2 with a gap from the outer front wall 41 of the outer case 40, and a pair of inner side walls 52 disposed to face each other in the rightward-leftward direction L3, and is formed in a frame shape having a C-shape in a plan view, which is open upward, downward, and rearward. In the illustrated example, an outer shape of the inner case 50 is formed so that the length along the forward-rearward direction L2 is longer than the length along the rightward-leftward direction L3 to correspond to the outer case 40.

The pair of inner side walls 52 is disposed inside the pair of outer side walls 43. In this manner, the whole inner case 50 is accommodated inside the outer case 40, and is movable in the forward-rearward direction L2 together with the outer case 40.

The inner front wall 51 pinches and fixes the connection piece 15 in the plunger 12 between the outer front wall 41 and the inner front wall 51. In this manner, a rear end portion of the plunger shaft 13 and the movable case 31 are integrally combined with each other and, are connected to each other. Therefore, the plunger shaft 13 and the movable case 31 are integrally movable in the forward-rearward direction L2.

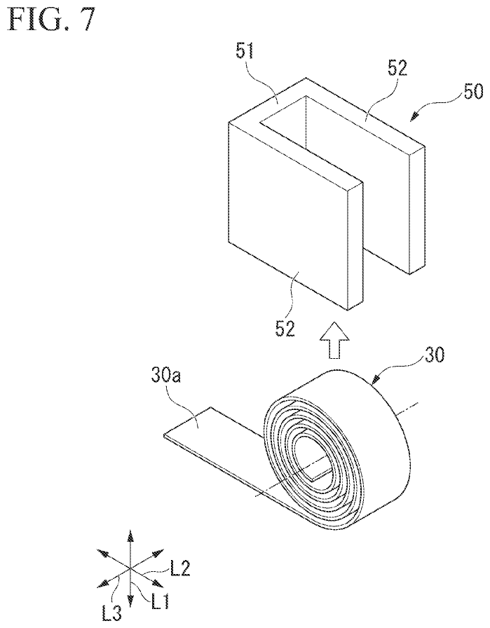

As illustrated in FIG. 7, the spiral spring 30 is formed by spirally winding a long strip-shaped material (for example, made of metal) having a thin thickness and a predetermined width. The spiral spring 30 is accommodated inside the inner case 50 in a posture in which a center line is parallel to the rightward-leftward direction L3. In this case, as illustrated in FIG. 6, an outer end portion 30a side of the spiral spring 30 is pulled out forward of the movable case 31 from below the inner case 50 and the outer case 40, and is connected to the holding base 21a in the holding member 21.

Therefore, an elastic restoring force acts on the spiral spring 30 so that the spiral spring 30 restores an original state by winding the outer end portion 30a side.

In the drive unit 22 configured as described above, the spiral spring 30 tries to restore and deform to the original state by winding the outer end portion 30a side. Accordingly, a winding portion accommodated inside the inner case 50 of the spiral spring 30 can be moved forward by the elastic restoring force. In this manner, the whole movable case 31 can be moved forward, and the plunger 12 is moved forward by the driving force F1 generated by the elastic restoring force of the spiral spring 30 so that the plunger 12 is pushed into the syringe 10.

For example, compared to a coil spring, the spiral spring 30 has a characteristic that the elastic restoring force is substantially constant while the spiral spring 30 restores the original state by being wound from a stretched state, and thus, can be suitably used as a so-called constant load spring. Therefore, in the present embodiment, the plunger 12 can be pressed with the predetermined constant driving force (constant pressure driving force) F1.

As illustrated in FIG. 2, the adjustment mechanism 23 is a mechanism that applies an auxiliary force F2 (positive thrust) to the plunger 12 in a forward direction or a reaction force F3 (negative thrust) to the plunger 12 in a rearward direction, in accordance with a difference between a resistance force generated by the movement of the plunger 12 inside the syringe 10 and the driving force F1 generated by the drive unit 22.

The configuration will be described in detail below.

As illustrated in FIGS. 1 to 4, the adjustment mechanism 23 includes a feed screw (movable body according to the present invention) 60 disposed to be movable in conjunction with the plunger 12, a feed mechanism 61 that moves the feed screw 60 forward at a predetermined speed and applies the auxiliary force F2 to the plunger 12 via the feed screw 60, and a braking unit 63 that applies the reaction force F3 to the plunger 12 in the rearward direction via the feed screw 60.

The feed screw 60 is disposed on the rear side of the movable case 31, and is disposed to be movable in the forward-rearward direction L2 along a first axis (axis according to the present invention) O1 disposed coaxially with the syringe axis R.

A male screw portion 60a is formed over the entire length on the outer peripheral surface of the feed screw 60. Furthermore, a front end portion of the feed screw 60 is integrally combined with the outer rear wall 42 of the outer case 40 via a connection nut 65. Therefore, the feed screw 60 is connected in series to the plunger 12 via the movable case 31, and is movable in the forward-rearward direction L2 in conjunction with the plunger 12.

Furthermore, the feed screw 60 is combined with the movable case 31 so that the rotation around a first axis O1 is restricted.

The feed screw 60 configured as described above is guided by a first guide member 72 provided in a first support portion 70 erected on the upper surface of the base plate 20, and a second guide member 82 provided in a second support portion 80 erected on the upper surface of the base plate 20.

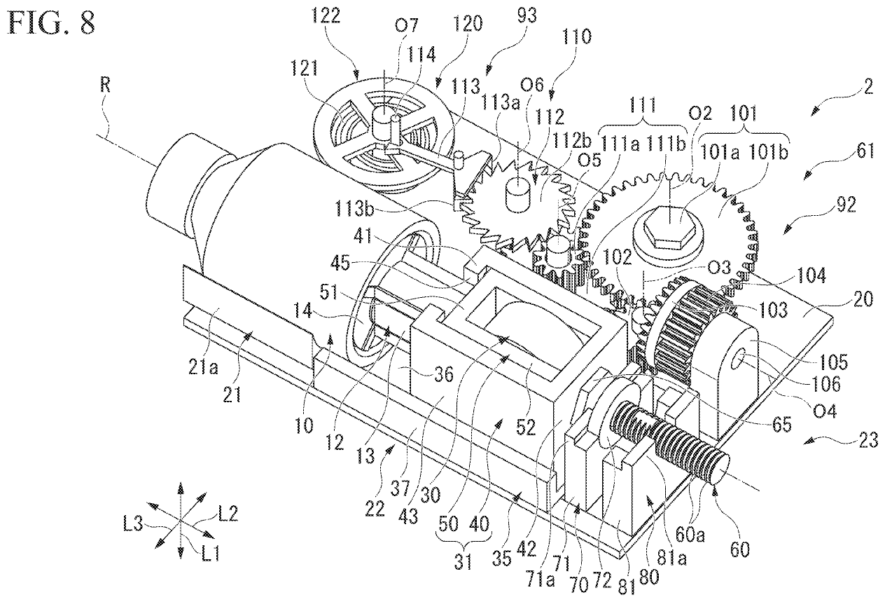

As illustrated in FIG. 8, the first support portion 70 includes a first support base 71 disposed on the rear side of the outer rear wall 42 in the outer case 40, a first guide member 72 held by the first support base 71, and a first cover 73 that presses the first guide member 72 from above.

The first support base 71 holds the first guide member 72 from below to be detachable upward. In addition, a first restriction wall 71a that comes into contact with the first guide member 72 from behind is formed at the upper end portion of the first support base 71. Therefore, the first guide member 72 is held by the first support base 71 in a state where rearward movement is restricted by the first restriction wall 71a. The first guide member 72 is formed in an annular shape having an insertion hole into which the feed screw 60 is inserted, and guides the feed screw 60.

For example, the first guide member 72 is a ball bearing having an inner ring, an outer ring, and a plurality of balls. However, the first guide member 72 is not limited to the ball bearing. In each drawing, the first guide member 72 is illustrated in a simplified manner.

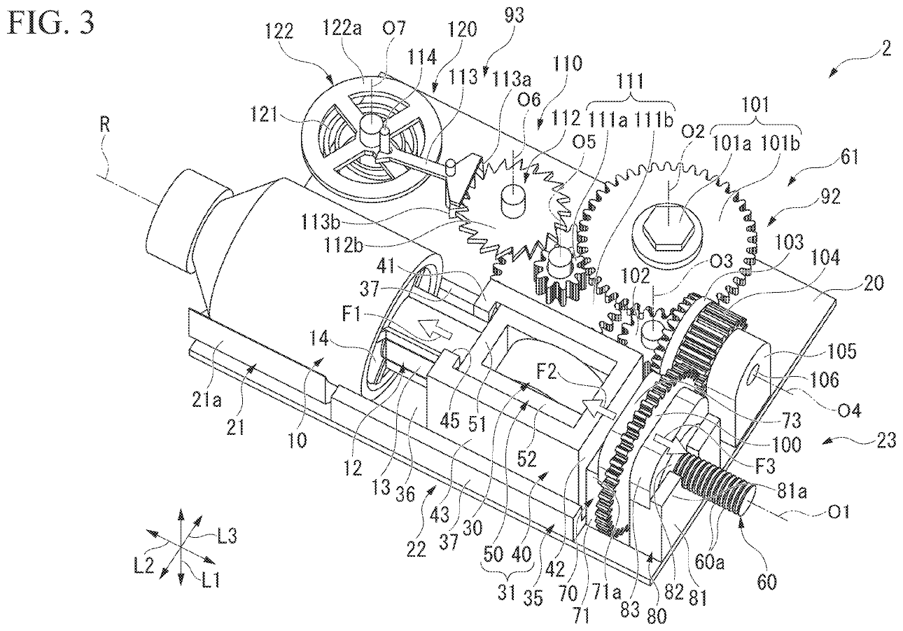

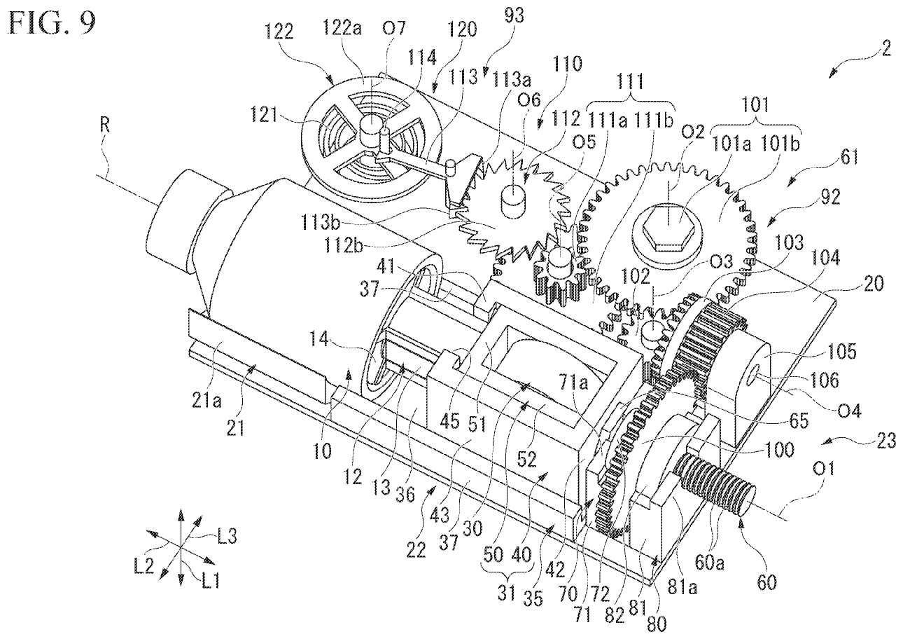

As illustrated in FIGS. 3 and 9, the second support portion 80 includes a second support base 81 disposed on the rear side of the first support base 71 in a state of having an interval from the first support base 71, a second guide member 82 held by the second support base 81, and a second cover 83 that presses the second guide member 82 from above.

The second support base 81 holds the second guide member 82 from below to be detachable upward. In addition, a second restriction wall 81a that comes into contact with the second guide member 82 from the front is formed in an upper end portion of the second support base 81. Therefore, the second guide member 82 is held by the second support base 81 in a state where forward movement is restricted by the second restriction wall 81a. The second guide member 82 is formed in an annular shape having an insertion hole into which the feed screw 60 is inserted, and guides the feed screw 60.

For example, the second guide member 82 is a ball bearing having an inner ring, an outer ring, and a plurality of balls. However, the second guide member 82 is not limited to the ball bearing. In each drawing, the second guide member 82 is illustrated in a simplified manner.

The feed screw 60 is configured as described above. Accordingly, for example, when assembled, the feed screw 60 is integrally combined with the outer case 40 in the movable case 31 by using a connection nut 65, and the first guide member 72 and the second guide member 82 are attached to the feed screw 60. Thereafter, the first guide member 72 is incorporated into the first support base 71 from above, and the movable case 31 is set on the guide plate 35 while the second guide member 82 is incorporated into the second support base 81 from above. Thereafter, the first cover 73 is combined with the first support base 71, and the second cover 83 is combined with the second support base 81. In this manner, the movable case 31 and the feed screw 60 that are integrally combined with each other can be assembled to each other.

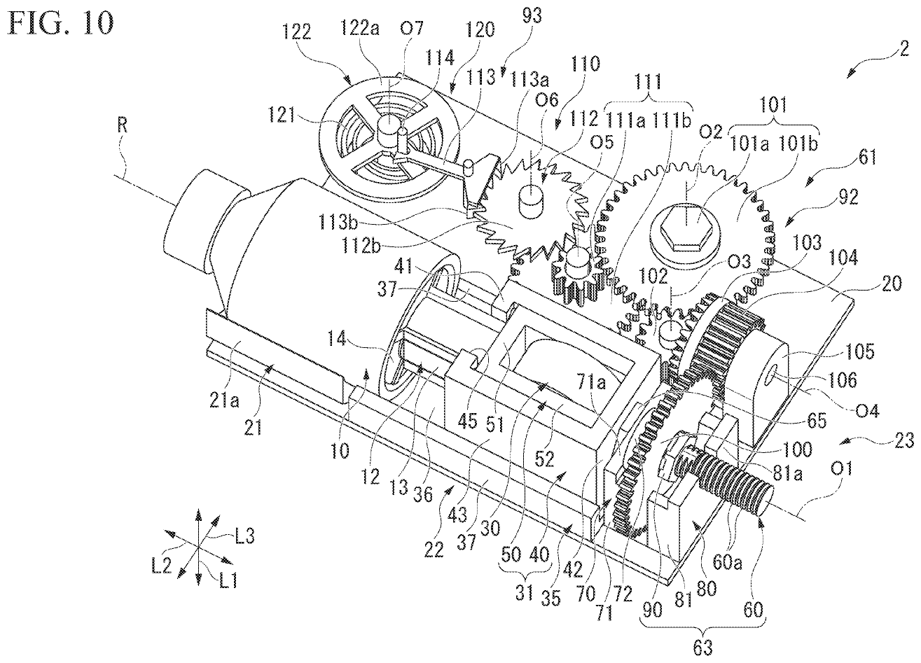

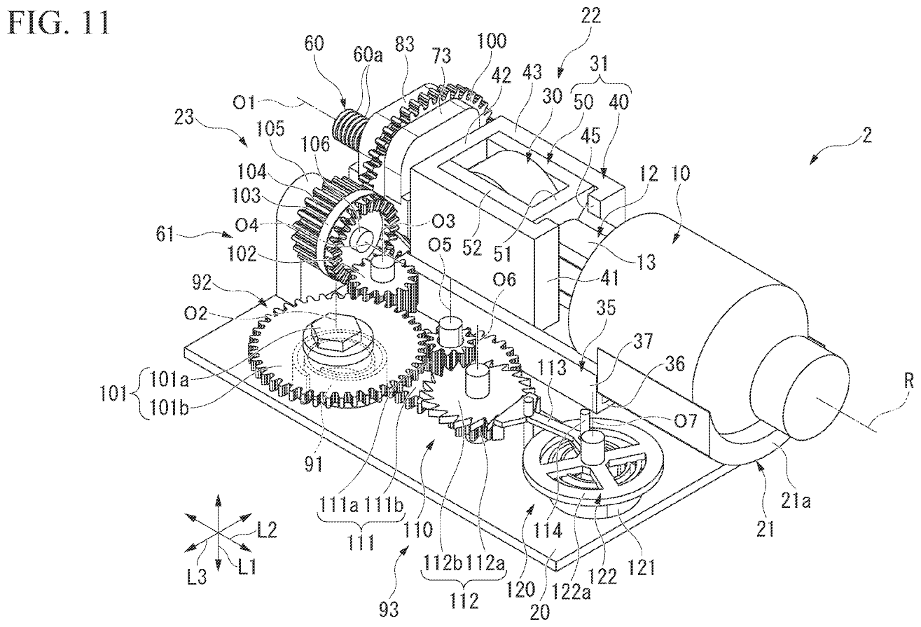

As illustrated in FIGS. 10 and 11, the feed mechanism 61 has a female screw portion (not illustrated) screwed to the male screw portion 60a of the feed screw 60, and includes a nut member 90 screwed to the feed screw 60, a mainspring (drive source according to the present invention) 91 that generates power (rotational torque) for rotating the nut member 90, a train wheel mechanism 92 that transmits the power from the mainspring 91 to the nut member 90, and a speed control mechanism 93 that controls a speed of the train wheel mechanism 92.

As illustrated in FIG. 10, the nut member 90 is screwed to a portion located between the first support portion 70 and the second support portion 80 in the feed screw 60, and is integrally formed with a feed wheel 100 disposed to be rotatable around the first axis O1. In this manner, the nut member 90 can rotate around the first axis O1 in association with the rotation of the feed wheel 100.

In the illustrated example, as an example, the nut member 90 is disposed between the feed wheel 100 and the second support portion 80. However, the present invention is not limited to this case, and the nut member 90 may be provided between the feed wheel 100 and the first support portion 70.

In particular, the nut member 90 and the feed wheel 100 are disposed to be pinched between the first support portion 70 and the second support portion 80, thereby restricting the movement in the forward-rearward direction L2.

As illustrated in FIGS. 10 and 11, the train wheel mechanism 92 includes a drive wheel 101 rotating around a second axis 02 by the power generated from the mainspring 91, a first intermediate wheel 102 rotating around a third axis O3 in association with the rotation of the drive wheel 101, a bevel wheel 103 rotating around a fourth axis O4 in association with the rotation of the first intermediate wheel 102, and a second intermediate wheel 104 rotating around the fourth axis O4 together with the bevel wheel 103.

However, the number or a disposition relationship of the wheels forming the train wheel mechanism 92 is not limited to this case, and may be changed as appropriate.

The drive wheel 101 is disposed on the upper surface of the base plate 20 in a state where the second axis O2 is parallel to the upward-downward direction L1. In the illustrated example, the drive wheel 101 is disposed at a position separated from the guide plate 35 in the rightward-leftward direction L3. The drive wheel 101 includes a drive shaft portion 101a and a drive gear 101b integrally formed with the drive shaft portion 101a. The mainspring 91 is disposed below the drive gear 101b.

The mainspring 91 is equivalent to that used in a mechanical timepiece, is formed in a spiral shape, and can generate the power by an unwinding operation.

The mainspring 91 is accommodated in an accommodation portion (not illustrated) such as a barrel complete in the mechanical timepiece, and an outer end portion thereof is attached to the inside of the accommodation portion. The inner end portion of the mainspring 91 is locked to the drive shaft portion 101a. In this manner, the drive shaft portion 101a is rotated around the second axis O2 so that the mainspring 91 can be wound to reduce the diameter.

Furthermore, the inner end portion of the mainspring 91 is locked to the drive shaft portion 101a. Accordingly, the whole drive wheel 101 can be rotated around the second axis O2 by unwinding the mainspring 91 to enlarge the diameter.

A clutch mechanism (not illustrated) such as a one-way clutch is provided between the drive shaft portion 101a and the drive gear 101b. In the clutch mechanism, when the drive shaft portion 101a is rotated in a winding direction of the mainspring 91, the drive shaft portion 101a is idled with respect to the drive gear 101b. When the drive shaft portion 101a is rotated in association with an unwinding operation of the mainspring 91, the drive gear 101b and the drive shaft portion 101a are rotated together. In this manner, the drive gear 101b can rotate only when the mainspring 91 is unwound.

The first intermediate wheel 102 is disposed on the upper surface of the base plate 20 in a state where the third axis O3 is parallel to the upward-downward direction L1. In the illustrated example, the first intermediate wheel 102 is located on the rear side of the drive shaft portion 101a, and is disposed to be located between the guide plate 35 and the drive wheel 101. The first intermediate wheel 102 meshes with the drive gear 101b of the drive wheel 101. In this manner, the first intermediate wheel 102 can rotate around the third axis O3 in association with the drive wheel 101.

The bevel wheel 103 is rotatably attached to a rotary shaft portion 106 fixed to a pedestal 105 erected on the upper surface of the base plate 20. The pedestal 105 is disposed to be adjacent to the first support portion 70 at an interval in the rightward-leftward direction L3. The rotary shaft portion 106 is disposed to be parallel to the forward-rearward direction L2, and is supported by the pedestal 105 in a cantilevered manner. A center line of the rotary shaft portion 106 is the fourth axis O4.

The bevel wheel 103 is rotatably attached to the rotary shaft portion 106 in a state of meshing with the first intermediate wheel 102. In this manner, the bevel wheel 103 can rotate around the fourth axis O4 in association with the rotation of the first intermediate wheel 102.

The second intermediate wheel 104 is rotatably attached to the rotary shaft portion 106 in a state of being integrally combined with the bevel wheel 103. Therefore, the second intermediate wheel 104 can rotate together around the fourth axis O4 in association with the rotation of the bevel wheel 103. Then, the second intermediate wheel 104 meshes with the feed wheel 100.

The train wheel mechanism 92 is configured as described above. Accordingly, the power generated by the unwinding operation of the mainspring 91 can be transmitted to the feed wheel 100 via the drive wheel 101, the first intermediate wheel 102, the bevel wheel 103, and the second intermediate wheel 104, and the feed wheel 100 can be rotated around the first axis O1 together with the nut member 90.

As illustrated in FIGS. 10 and 11, the speed control mechanism 93 includes an escapement 110 that controls the rotation of the above-described train wheel mechanism 92, and a speed controller 120 that controls the speed of the escapement 110. The escapement 110 and the speed controller 120 have configurations the same as those generally used for the mechanical timepiece. Therefore, detailed description of the escapement 110 and the speed controller 120 will be omitted.

The escapement 110 includes an intermediate wheel 111 rotating around a fifth axis O5 in association with the rotation of the drive wheel 101, and an escape wheel 112 rotating around a sixth axis O6 in association with the rotation of the intermediate wheel 111, and a pallet fork 113 that allows an escape of the escape wheel 112 to rotate regularly, and can control the train wheel mechanism 92 by using regular vibrations from a balance with hairspring 122 (to be described later).

The intermediate wheel 111 is disposed on the upper surface of the base plate 20 in a state where the fifth axis O5 is parallel to the upward-downward direction L1. In the illustrated example, the intermediate wheel 111 is located on the front side of the drive shaft portion 101a, and is disposed to be located between the guide plate 35 and the drive wheel 101. The intermediate wheel 111 has an intermediate pinion 111a meshing with the drive gear 101b in the drive wheel 101, and an intermediate gear 111b. In this manner, the intermediate wheel 111 can rotate around the fifth axis O5 in association with the drive wheel 101.

The intermediate wheel 111 is not essential, and may not be provided. For example, the escape wheel 112 may be configured to rotate in association with the drive wheel 101.

The escape wheel 112 is disposed on the front side of the intermediate wheel 111 in a state where the fifth axis O5 is parallel to the upward-downward direction L1. The escape wheel 112 includes an escape pinion 112a meshing with the intermediate gear 111b and an escape gear 112b having a plurality of escape teeth. In this manner, the escape wheel 112 can rotate around the sixth axis O6 in association with the rotation of the intermediate wheel 111.

The pallet fork 113 has an entry pallet 113a and an exit pallet 113b which are disposed on the front side of the escape wheel 112, can pivot (can oscillate) based on reciprocating rotation of the balance with hairspring 122, and can engage with and disengage from escape teeth in the escape wheel 112. The entry pallet 113a and the exit pallet 113b can alternately engage with and disengage from the escape teeth in association with pivoting of the pallet fork 113.

Therefore, the entry pallet 113a and the exit pallet 113b alternately engage with and disengage from the escape teeth in association with the pivoting of the pallet fork 113. In this manner, the rotation of the escape wheel 112 can be controlled, and the power transmitted to the escape wheel 112 can be transmitted to the balance with hairspring 122 via the pallet fork 113 and an impulse pin 114 so that the balance with hairspring 122 can be replenished with rotational energy.

The speed controller 120 includes a hairspring 121 and a balance with hairspring 122 that performs reciprocating rotation (forward and reverse rotation) around a seventh axis O7 with a steady amplitude (oscillation angle) by using the hairspring 121 as a power source.

The balance with hairspring 122 is disposed on the front side of the pallet fork 113 in a state where the seventh axis O7 is parallel to the upward-downward direction L1. The balance with hairspring 122 includes a balance wheel 122a disposed coaxially with the seventh axis O7.

The hairspring 121 is formed in a spiral shape around the seventh axis O7, and is elastically deformable to enlarge and reduce the diameter. The inner end portion of the hairspring 121 is locked to the balance with hairspring 122 via a double roller (not illustrated). In this manner, the balance with hairspring 122 can perform reciprocating rotation around the seventh axis O7 by using the hairspring 121 as a power source.

The speed control mechanism 93 having the escapement 110 and the speed controller 120 which are configured as described above are provided. Accordingly, the power generated by the unwinding operation of the mainspring 91 is used so that the feed wheel 100 can be rotated around the first axis O1 at a predetermined rotation speed.

In this manner, the feed screw 60 whose rotation around the first axis O1 is restricted can be moved forward at a predetermined speed, and the auxiliary force F2 can be applied to the plunger 12 via the movable case 31.

The nut member 90 is screwed to the feed screw 60, and the male screw portion 60a and the female screw portion mesh with each other. Therefore, for example, when the feed screw 60 is pulled forward by the drive unit 22, the male screw portion 60a of the feed screw 60 is in a state of being pressed forward with respect to the female screw portion of the nut member 90. Therefore, in addition to a meshing force between the gears in the above-described train wheel mechanism 92, a meshing force of the nut member 90 and the feed screw 60 can be used as the reaction force F3.

Therefore, as illustrated in FIGS. 2 and 10, at least the male screw portion 60a of the feed screw 60 and the female screw portion of the nut member 90 function as the braking unit 63 that applies the reaction force F3 to the plunger 12 in the rearward direction via the feed screw 60.

### Operation of Chemical Solution Administration Device

Next, a case will be described where the chemical solution W is administered into the user's body by using the chemical solution administration device 1 configured as described above.

As an initial state in this case, as illustrated in FIG. 1, the chemical solution administration device 1 is mounted on the user's body surface S, and the indwelling needle 4 indwells the body surface S in a state of puncturing into the body. Furthermore, the syringe 10 filled with the chemical solution W is set in the holding member 21 of the chemical solution pump 2. Furthermore, the movable case 31 is located on the rear side of the guide plate 35, the plunger 12 is set at a pushing start position, and the mainspring 91 is properly wound to accumulate power.

When the chemical solution W starts to be administered in the above-described initial state, as illustrated in FIGS. 2 and 3, the plunger 12 can be pressed forward by the drive unit 22 with the predetermined driving force F1 (predetermined constant driving force F1), and the plunger 12 can be pressed forward via the movable case 31 by moving the feed screw 60 forward at a predetermined speed.

This configuration will be described in detail.

First, the spiral spring 30 in the drive unit 22 tries to restore and deform to the original state by winding the outer end portion 30a side. In this case, the outer end portion 30a side of the spiral spring 30 is connected to the holding base 21a. Accordingly, the winding portion side accommodated inside the inner case 50 in the spiral spring 30 can be moved forward by the elastic restoring force by using the outer end portion 30a side as a base point. In this manner, the whole movable case 31 can be moved forward, and the plunger 12 can be moved forward by the driving force F1 generated by the elastic restoring force of the spiral spring 30.

As a result, the plunger 12 can be pushed into the syringe 10, and the chemical solution W inside the syringe 10 can be discharged to the indwelling needle 4 side.

In particular, when the plunger 12 is pressed by using the driving force F1, the adjustment mechanism 23 applies the auxiliary force F2 in a forward direction in which the plunger 12 moves into the syringe 10, or applies the reaction force F3 in a rearward direction opposite thereto in accordance with a difference between the resistance force generated by the movement of the plunger 12 inside the syringe 10 and the driving force F1.

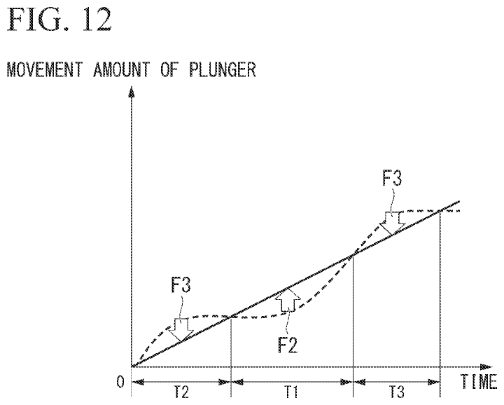

For example, when the plunger 12 is pressed forward by using the constant driving force F1, it is preferable that a movement speed per unit time is ideally constant as illustrated by a solid line illustrated in FIG. 12, and a movement amount of the plunger 12 has linearity.

However, the resistance force (for example, sliding resistance generated between the syringe 10 and the plunger 12 or viscous resistance of the chemical solution W) actually generated by the movement of the plunger 12 is changed. Accordingly, as indicated by a dotted line illustrated in FIG. 12, even when the plunger 12 is pressed with the constant driving force F1, a movement speed per unit time is changed corresponding to the resistance force, and the movement amount is changed.

That is, when the resistance force is great, the movement speed of the plunger 12 becomes slower, and the movement amount per unit time of the plunger 12 decreases as indicated in a section T1 illustrated in FIG. 12. On the other hand, when the resistance force is weak, the movement speed of the plunger 12 becomes faster, and the movement amount per unit time of the plunger 12 increases as indicated in sections T2 and T3 illustrated in FIG. 12.

In the present embodiment, the adjustment mechanism 23 is provided. Accordingly, in the above-described section T1, the auxiliary force F2 (that is, positive thrust) is applied to the plunger 12 in the forward direction. In this manner, it is possible to increase the movement speed of the plunger 12. On the other hand, in the above-described sections T2 and T3, the reaction force F3 (that is, negative thrust) is applied to the plunger 12 in the rearward direction. In this manner, it is possible to prevent the movement speed of the plunger 12 from becoming faster.

In this way, the adjustment mechanism 23 can correct the movement speed to offset the influence of the resistance force by applying the auxiliary force F2 or the reaction force F3 to the plunger 12, in accordance with a difference between the resistance force generated by the movement of the plunger 12 inside the syringe 10 and the driving force F1. Therefore, the plunger 12 can be moved by the driving force F1 applied from the drive unit 22.

As a result, as indicated by a solid line illustrated in FIG. 12, it is possible to move the plunger 12 with a constant movement amount (constant amount), and the chemical solution W can be accurately discharged from the syringe 10 with a constant discharge amount (constant amount). Therefore, it is possible to provide the chemical solution pump 2 having excellent discharge accuracy.

Therefore, according to the chemical solution administration device 1 of the present embodiment including the chemical solution pump 2, a determined amount of the chemical solution W can be periodically administered into the body through the indwelling needle 4, for example.

For example, the driving force F1 and the auxiliary force F2 can be appropriately adjusted by adjusting a curvature of the spiral spring 30 or a reduction ratio of the train wheel mechanism 92. Accordingly, depending on a use or a type of the chemical solution W, the movement speed of the plunger 12 can be changed when the chemical solution W is administered.

As described above, according to the chemical solution administration device 1 and the chemical solution pump 2 of the present embodiment, the chemical solution W can be accurately discharged with the constant discharge amount. Therefore, for example, to the chemical solution administration device 1 and the chemical solution pump 2 can be suitably used as an insulin pump and an insulin administration device which are excellent in discharge accuracy and discharge reliability.

A case will be described in detail where the auxiliary force F2 and the reaction force F3 are applied to the plunger 12 by using the feed screw 60.

As illustrated in FIG. 11, when the power is generated by the unwinding operation the mainspring 91, the drive wheel 101 is rotated by the power. Accordingly, the first intermediate wheel 102, the bevel wheel 103, and the second intermediate wheel 104 can be sequentially rotated in association with the rotation of the drive wheel 101, and finally, the feed wheel 100 can be rotated. In this manner, the feed wheel 100 can be rotated around the first axis O1 together with the nut member 90. The rotation around the first axis O1 is restricted. Accordingly, the feed screw 60 is not rotated together with the rotation of the nut member 90. Therefore, the feed screw 60 can be moved forward along the first axis O1 in association with the rotation of the nut member 90.

In this case, the speed of the train wheel mechanism 92 is controlled by the speed control mechanism 93 having the escapement 110 and the speed controller 120. Accordingly, the power generated by the unwinding operation of the mainspring 91 is used so that the feed wheel 100 and the nut member 90 can be rotated around the first axis O1 at a predetermined rotation speed. Therefore, the feed screw 60 can be moved forward at a predetermined speed.

The feed screw 60 is moved forward at the predetermined speed in this way. Accordingly, the auxiliary force F2 can be applied to the plunger 12 via the movable case 31.

Furthermore, for example, when the resistance force generated by the movement of the plunger 12 is weak and the feed screw 60 is pulled forward by the resistance force, the male screw portion 60a of the feed screw 60 is in a state of being pressed forward with respect to the female screw portion of the nut member 90. Therefore, in addition to the meshing force between the gears in the train wheel mechanism 92, the meshing force of the feed screw 60 and the nut member 90 can be used to apply the reaction force F3 to the plunger 12 in the rearward direction. Accordingly, it is possible to prevent the movement speed of the plunger 12 from becoming faster.

In particular, according to the chemical solution pump 2 and the chemical solution administration device 1 of the present embodiment, the driving force F1 is generated by using the spiral spring 30, and the driving force for rotating the nut member 90 is generated by the unwinding operation of the mainspring 91. Therefore, electric power of a battery is not needed to discharge the chemical solution W. Therefore, since the electric power is not used, low cost can be easily achieved, and safety can be improved.

Furthermore, the drive unit 22 can have a simple configuration using the spiral spring 30. Therefore, the drive unit 22 easily achieves low cost and a simplified configuration in this regard.

### Second Embodiment

Next, a second embodiment of a chemical solution pump according to the aspect of the present invention will be described with reference to the drawings. In the second embodiment, the same reference numerals will be assigned to configuration elements the same as configuration elements in the first embodiment, and description thereof will be omitted.

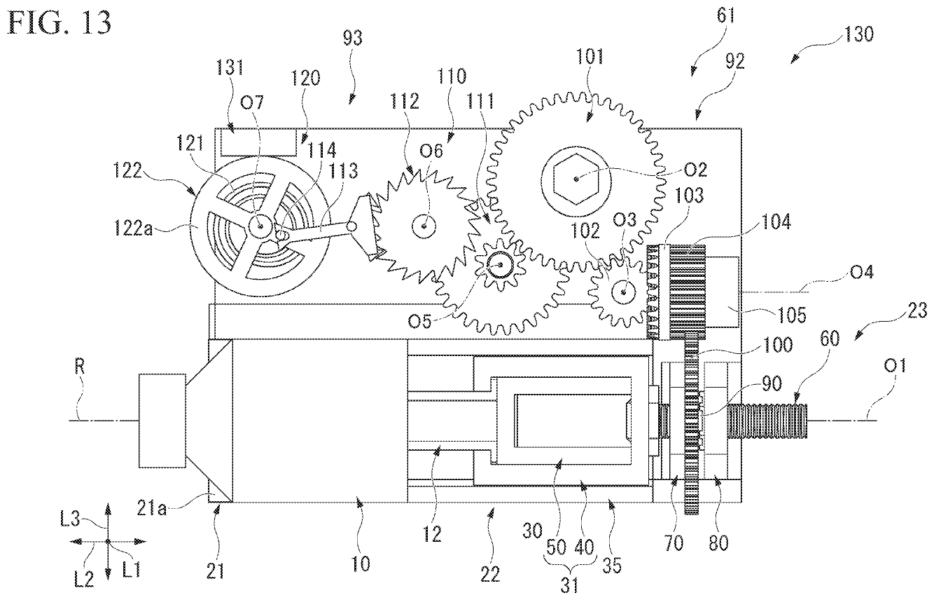

As illustrated in FIG. 13, a chemical solution pump 130 of the present embodiment includes a switching mechanism 131 that switched between stopping and starting transmission of power from the mainspring 91 to the nut member 90.

The switching mechanism 131 is a mechanism for adding a so-called start and stop function, and is provided in an intermediate portion of a transmission route in which the power generated by the mainspring 91 is transmitted to the nut member 90. In the present embodiment, the switching mechanism 131 is provided adjacent to the balance with hairspring 122 forming the speed controller 120.

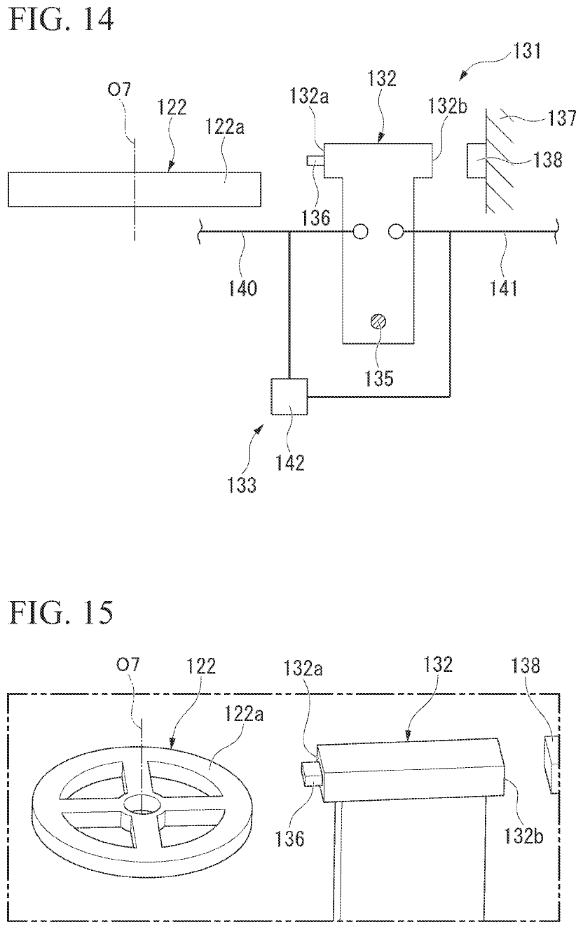

As illustrated in FIGS. 14 and 15, the switching mechanism 131 includes an oscillator plate 132 disposed adjacent to the balance with hairspring 122 and supported on the upper surface of the base plate 20 to be capable of oscillating, and an operation unit 133 that controls oscillation of the oscillator plate 132.

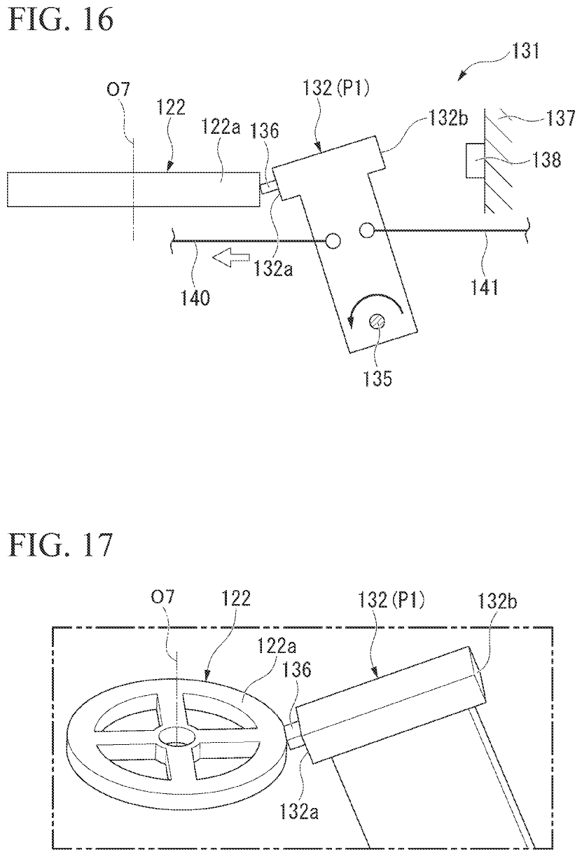

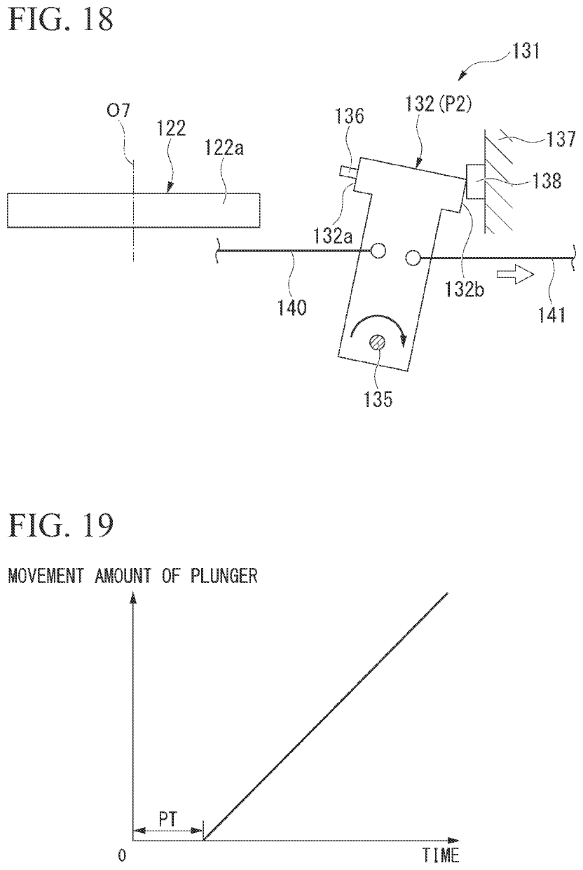

For example, the oscillator plate 132 is formed of a metal material, and is a magnetic body. The oscillator plate 132 is formed to extend along the upward-downward direction L1, and an oscillating shaft portion 135 is provided on a lower end portion side. The oscillator plate 132 can oscillate to reciprocate between a stop position P1 (locking position) illustrated in FIGS. 16 and 17 which is close to the balance with hairspring 122 around the oscillating shaft portion 135 and a start position P2 (unlocking position) illustrated in FIG. 18 which is separated from the balance with hairspring 122.

In FIGS. 13 to 18, the balance with hairspring 122 is simplified in the illustration, and the balance wheel 122a is mainly illustrated.

As illustrated in FIGS. 14 and 15, an upper end portion of the oscillator plate 132 has a first operation surface 132a facing the balance wheel 122a side and a second operation surface 132b facing a side opposite to the first operation surface 132a. A first magnet 136 that attracts the balance wheel 122a when the oscillator plate 132 is located at the stop position P1 is attached to the first operation surface 132a.

Furthermore, the base plate 20 is provided with a holding wall portion 137 to be located on a side opposite to the balance with hairspring 122 across the oscillator plate 132. A second magnet 138 that attracts the second operation surface 132b when the oscillator plate 132 is located at the start position P2 is attached to the holding wall portion 137.

The oscillator plate 132 is configured as described above. Accordingly, when the oscillator plate 132 is located at the stop position P1, the rotation of the balance with hairspring 122 can be stopped by a magnetic force acting between the first magnet 136 and the balance wheel 122a, and a state where the oscillator plate 132 is positioned at the stop position P1 can be maintained.

Furthermore, when the oscillator plate 132 is located at the start position P2, a state where the oscillator plate 132 is positioned at the start position P2 can be maintained by a magnetic force acting between the second magnet 138 and the second operation surface 132b.

As illustrated in FIG. 14, the operation unit 133 has a role of causing the oscillator plate 132 to oscillate around the oscillating shaft portion 135 so that the oscillator plate 132 is located at either the stop position P1 or the start position P2. FIGS. 14 and 15 illustrate a transition state where the oscillator plate 132 is located between the stop position P1 and the start position P2.

The operation unit 133 includes a first shape memory alloy wire 140 and a second shape memory alloy wire 141 which are connected to the oscillator plate 132, and a control unit 142 that controls energizing by applying a predetermined pulse voltage to the first shape memory alloy wire 140 and the second shape memory alloy wire 141.

For example, the first shape memory alloy wire 140 and the second shape memory alloy wire 141 are wires made of nickel-titanium alloy, and are wires that instantaneously shrink when energized and heated, and that stretch when heat is radiated.

One end portion side of the first shape memory alloy wire 140 is connected to the oscillator plate 132, and shrinks to pull the oscillator plate 132 to the stop position P1 so that the oscillator plate 132 can be shifted to the stop position P1. One end portion side of the second shape memory alloy wire 141 is connected to the oscillator plate 132, and shrinks to pull the oscillator plate 132 to the start position P2 so that the oscillator plate 132 can be shifted to the start position P2.

The first shape memory alloy wire 140 and the second shape memory alloy wire 141 are electrically connected to the control unit 142, and a predetermined voltage is individually applied from the control unit 142. In addition, the oscillator plate 132 is formed of a material having higher thermal conductivity than that of the first shape memory alloy wire 140 and the second shape memory alloy wire 141. In this manner, the oscillator plate 132 also functions as a heat radiating body that radiates heat from the first shape memory alloy wire 140 and the second shape memory alloy wire 141. Therefore, the first shape memory alloy wire 140 and the second shape memory alloy wire 141 can shrink by heating and thereafter, can quickly be radiated to release a shrunk state, and can be shifted to a stretched state (loosened state).

For example, the control unit 142 can instantaneously apply a pulse voltage to each of the first shape memory alloy wire 140 and the second shape memory alloy wire 141 to perform rapid heating. In this manner, the oscillator plate 132 can oscillate at any desired timing to be moved to the stop position P1 or the start position P2.

### Operation of Chemical Solution Pump

According to the chemical solution pump 130 of the present embodiment configured as described above, it is possible to achieve operational effects the same as those of the first embodiment, and in addition, the following operational effects can be further achieved.

That is, in a case of the chemical solution pump 130 of the present embodiment, the switching mechanism 131 can be used to switch between stopping and starting the power transmission from the mainspring 91 to the nut member 90. Accordingly, for example, the nut member 90 can be rotated at any desired timing, and a time for rotating the nut member 90 can be adjusted. In particular, the rotation of the nut member 90 can be stopped to stop the movement of the feed screw 60 itself provided in conjunction with the plunger 12. Accordingly the movement of the plunger 12 into the syringe 10 can be stopped.

Therefore, it is possible to adjust a discharge timing or a discharge time of the chemical solution W from the syringe 10. Accordingly, it is possible to provide the chemical solution pump 130 which achieves convenient use and excellent discharge performance.

This configuration will be described in detail.

In the initial state, as illustrated in FIGS. 16 and 17, the oscillator plate 132 is located at the stop position P1. In this manner, the rotation of the whole balance with the hairspring 122 can be restricted by a magnetic force between the first magnet 136 and the balance wheel 122a. Therefore, the rotation of the drive wheel 101 can be restricted, and similarly, the rotation of the feed wheel 100 and the nut member 90 can be restricted. In this manner, it is possible to maintain a state where the operation of the chemical solution pump 130 is stopped.

Therefore, for example, a state where chemical solution administration is stopped can be maintained while the main body case 3 is mounted on the body surface S.

When the chemical solution administration starts from the above-described initial state, the control unit 142 instantaneously applies a pulse voltage to the second shape memory alloy wire 141 to rapidly heat the second shape memory alloy wire 141. In this manner, as illustrated in FIG. 18, the second shape memory alloy wire 141 can shrink, and the oscillator plate 132 can oscillate from the stop position P1 to the start position P2. In this manner, the first magnet 136 can be separated from the balance wheel 122a, and the oscillator plate 132 can be positioned at the start position P2 by the magnetic force between the second magnet 138 and the second operation surface 132b.

After the oscillator plate 132 is positioned at the start position P2 by the magnetic force, the second shape memory alloy wire 141 radiates the heat. Accordingly, the second shape memory alloy wire 141 is shifted from a shrunk state to a loosened state.

The oscillator plate 132 is located at the start position P2, and the first magnet 136 is separated from the balance wheel 122a. In this manner, the rotation of the balance with hairspring 122 can be started by the power of the hairspring 121, and the rotation of the drive wheel 101 can be started by the power of the mainspring 91. In this manner, the train wheel mechanism 92 can be operated in a state where the speed is controlled. Accordingly, the feed wheel 100 and the nut member 90 can be rotated to move the feed screw 60 forward at a predetermined speed.

As a result, as in the first embodiment, the plunger 12 can be moved forward based on the driving force F1 while the movement of the plunger 12 is adjusted by the adjustment mechanism 23, and the chemical solution W can be discharged with a constant discharge amount to be administered into the body.

In this way, according to the chemical solution pump 130 of the present embodiment, the operation can start at any desired timing. Therefore, for example, as illustrated in FIG. 19, the following method of use can be adopted. After a lapse of a predetermined time PT from when the main body case 3 is mounted, the plunger 12 starts to move so that the chemical solution W is continuously administered.

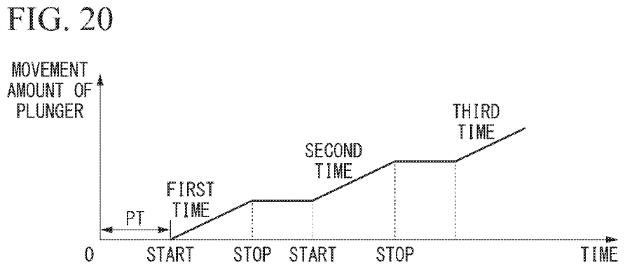

Furthermore, as illustrated in FIG. 20, the chemical solution W can be intermittently administrated instead of continuous administration of the chemical solution W.

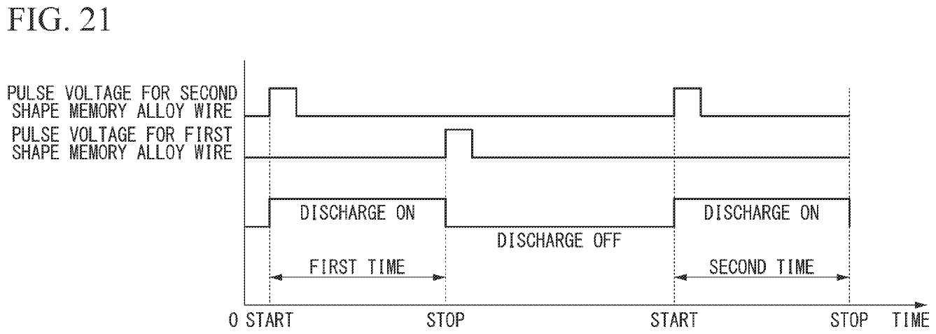

In this case, as illustrated in FIG. 21, from the initial state described above, the control unit 142 instantaneously applies a pulse voltage to the second shape memory alloy wire 141 so that the second shape memory alloy wire 141 shrinks. In this manner, the oscillator plate 132 can oscillate from the stop position P1 to the start position P2, and can be positioned at the start position P2. Accordingly, the rotation of the balance with hairspring 122 and the drive wheel 101 can start. In this manner, as described above, the feed screw 60 can be moved forward at a predetermined speed, and the chemical solution W can be administered with a constant discharge amount.

Subsequently, after a lapse of a prescribed administration time, the control unit 142 instantaneously applies a pulse voltage to the first shape memory alloy wire 140 so that the first shape memory alloy wire 140 shrinks. In this manner, the oscillator plate 132 can oscillate from the start position P2 to the stop position P1. Therefore, the rotation of the balance with hairspring 122 can be stopped by the magnetic force between the first magnet 136 and the balance wheel 122a, and the oscillator plate 132 can be positioned at the stop position P1. In this manner, the operation of the chemical solution pump 130 can be stopped by stopping the rotation of the balance with hairspring 122 and the drive wheel 101. Accordingly, the administration of the chemical solution W can be stopped.

Subsequently, after a lapse of a prescribed time for stopping the administration of the chemical solution, the administration of the above-described chemical solution W is repeatedly started and stopped. In this manner, as illustrated in FIGS. 20 and 21, discontinuous and intermittent administration can be performed by repeatedly administer the chemical solution with a time interval multiple times (second time and third time).

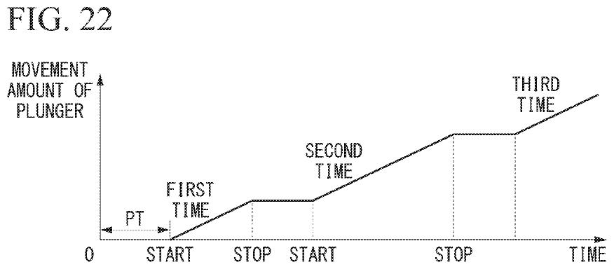

When the chemical solution is repeatedly administered with a time interval multiple times, for example, as illustrated in FIG. 22, the following method of use can be adopted. It is possible to perform irregularly administration in which the chemical solution W is irregularly administered, by lengthening a second administration time.

In this way, according to the chemical solution pump 130 of the present embodiment, there is provided the switching mechanism 131. Accordingly, it is possible to adjust a discharge timing or a discharge time of the chemical solution W from the syringe 10. Accordingly, it is possible to provide the chemical solution pump 130 which achieves convenient use and can be used in various ways corresponding to various purposes.

### Modification Example of Second Embodiment

In the above-described second embodiment, the second magnet 138 is attached to the holding wall portion 137 side. However, the present invention is not limited to this case. For example, as in the first magnet 136, the second magnet 138 may be attached to the second operation surface 132b of the oscillator plate 132. In this case, the holding wall portion 137 side may be the magnetic body.

Furthermore, in the above-described second embodiment, a configuration is adopted so that the rotation of the balance with hairspring 122 is restricted by using the magnetic force between the first magnet 136 and the balance wheel 122a. However, the present invention is not limited to a case of using the magnetic force. For example, the first magnet 136 may be omitted, the first operation surface 132a may be pressed against the outer peripheral surface of the balance wheel 122a, and a frictional force acting between the first operation surface 132a and the balance wheel 122a may be used to restrict the rotation of the balance with hairspring 122a.

Furthermore, in the above-described second embodiment, a configuration is adopted so that the oscillator plate 132 oscillates by using the stretching and shrinking property of the first shape memory alloy wire 140 and the second shape memory alloy wire 141. However, the configuration is not limited to this case. As long as the oscillator plate 132 can oscillate between the stop position P1 and the start position P2, the operation unit 133 may be appropriately configured without using the shape memory alloy wire.

Furthermore, in the above-described embodiment, a configuration is adopted so that the rotation of the balance with hairspring 122 is restricted and the restriction is released to switch between stopping and starting the power transmission from the mainspring 91 to the nut member 90. However, without being limited to a case of using the balance with hairspring 122, the switching mechanism 131 may be provided in an intermediate portion of a power transmission route to the nut member 90. Even in this case, the same operational effects can be achieved.

However, the balance with hairspring 122 is a component that rotates with a small rotational torque. Accordingly, a holding force required for restricting the rotation of the balance with hairspring 122 can be minimized. Therefore, it is possible to prevent the switching mechanism 131 itself from increasing in size, and it is easy to achieve a simplified configuration.

### Third Embodiment

Next, a third embodiment of a chemical solution pump according to the aspect of the present invention will be described with reference to the drawings. In the third embodiment, the same reference numerals will be assigned to configuration elements the same as configuration elements in the first embodiment, and description thereof will be omitted.

In the first embodiment, the speed control mechanism 93 controls the speed of the train wheel mechanism 92 by using the escapement 110 having the escape wheel 112 and the pallet fork 113, and the speed controller 120 having the balance with hairspring 122. However, in the present embodiment, the speed of the train wheel mechanism 92 is controlled by using an impeller.

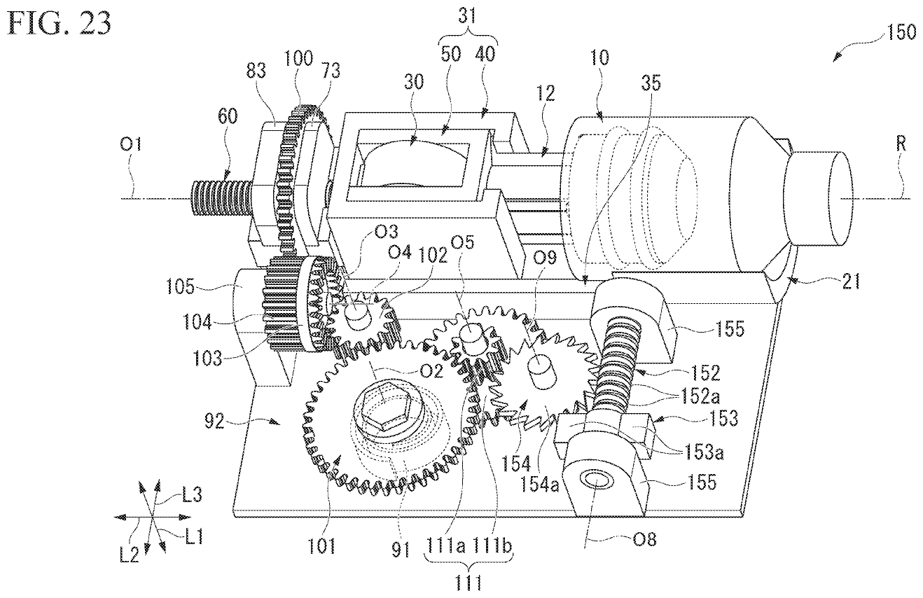

As illustrated in FIG. 23, in a chemical solution pump 150 of the present embodiment, the speed control mechanism 151 includes a worm shaft 152 disposed to be rotatable around an eighth axis O8, an impeller 153 rotating around the eighth axis O8 in association with the rotation of the worm shaft 152 and applying rotational resistance to the rotation of the worm shaft 152, and a worm wheel 154 rotating around a ninth axis O9 in association with the rotation of the intermediate wheel 111 and rotating the worm shaft 152.

The worm shaft 152 is disposed on the front side of the intermediate wheel 111, and is disposed on the upper surface of the base plate 20 so that the eighth axis O8 is parallel to the rightward-leftward direction L3. Both end portions of the worm shaft 152 are pivotally supported by a pair of bearing bases 155 fixed onto the base plate 20. In this manner, the worm shaft 152 can rotate stably around the eighth axis O8 with less rattling. A spiral worm groove 152a is formed on the outer peripheral surface of the worm shaft 152 over its entire length.

The impeller 153 is integrally combined with the worm shaft 152, and includes a pair of blade plates 153a. Therefore, the impeller 153 rotates while receiving air resistance from the impeller 153 in association with the rotation of the worm shaft 152. In this manner, the impeller 153 can apply rotational resistance to the rotation of the worm shaft 152. The number of blade plates 153a is not limited to one pair. The number may be one, or may be three or more.

The worm wheel 154 is disposed in place of the escape wheel 112 in the first embodiment, and is disposed on the upper surface of the base plate 20 in a state where the ninth axis O9 is parallel to the upward-downward direction L1. The worm wheel 154 includes a worm pinion (not illustrated) that meshes with the intermediate gear 111b, and a worm gear 154a that meshes with the worm groove 152a. In this manner, the worm wheel 154 can rotate around the ninth axis O9 in association with the rotation of the intermediate wheel 111, and can rotate the worm shaft 152 around the eighth axis O8.

### Operation of Chemical Solution Pump

The chemical solution pump 150 of the present embodiment configured as described above can also achieve operational effects as those of the first embodiment.

In particular, when the drive wheel 101 is rotated by the power generated by the unwinding operation of the mainspring 91, the intermediate wheel 111 and the worm wheel 154 can be rotated in association therewith. Therefore, the worm shaft 152 can be rotated in association with the rotation of the worm wheel 154, and the impeller 153 can be rotated. The impeller 153 receives air resistance corresponding to the rotation speed of the worm shaft 152 via the blade plate 153a. Accordingly, the rotation speed of the worm shaft 152 can be controlled to a constant speed, for example.

In this manner, even in a case of the present embodiment, the speed of the train wheel mechanism 92 can be controlled by using the air resistance of the impeller 153, and the power generated by the unwinding operation of the mainspring 91 is used so that the feed wheel 100 and the nut member 90 can be rotated around the first axis O1 at a predetermined rotation speed. Therefore, the feed screw 60 can be moved forward at a predetermined speed, and operational effects the same as that of the first embodiment can be achieved.

Furthermore, in a case of the first embodiment, a configuration is adopted so that the impulse pin 114 of the balance with hairspring 122 and the pallet fork 113 collide with each other when the speed of the train wheel mechanism 92 is controlled. Accordingly, collision sound is generated. In contrast, in a case of the present embodiment, the impeller 153 only rotates. Accordingly, only a so-called wind noise is generated, and the generated sound can be reduced, compared to the collision sound in the first embodiment. Therefore, it is possible to provide the chemical solution pump 150 which is excellently quiet.

### Modification Example of Third Embodiment

In the above-described third embodiment, the impeller 153 may be configured to rotate in a viscous fluid such as silicone oil having predetermined viscosity, for example. In this case, the impeller 153 can function as a so-called oil rotor, and can generate the rotational resistance (viscous resistance) corresponding to the rotation speed of the worm shaft 152. Therefore, even in this case, it is possible to achieve operational effects the same as those when the air resistance is used.

### Fourth Embodiment

Next, a fourth embodiment of a quantitative amount feed mechanism according to the aspect of the present invention will be described with reference to the drawings. In the fourth embodiment, the same reference numerals will be assigned to configuration elements the same as configuration elements in the first embodiment, and description thereof will be omitted.

In the first embodiment, the speed control mechanism 93 controls the speed of the train wheel mechanism 92 by using the escapement 110 having the escape wheel 112 and the pallet fork 113 and the speed controller 120 having the balance with hairspring 122. However, in the present embodiment, the speed of the train wheel mechanism is controlled by using an impeller. Furthermore, there is provided a switching mechanism that switches between stopping and starting the power transmission to the nut member 90 so that the rotation of the impeller is controlled by using the power generated in association with the unwinding operation of the switching mainspring.

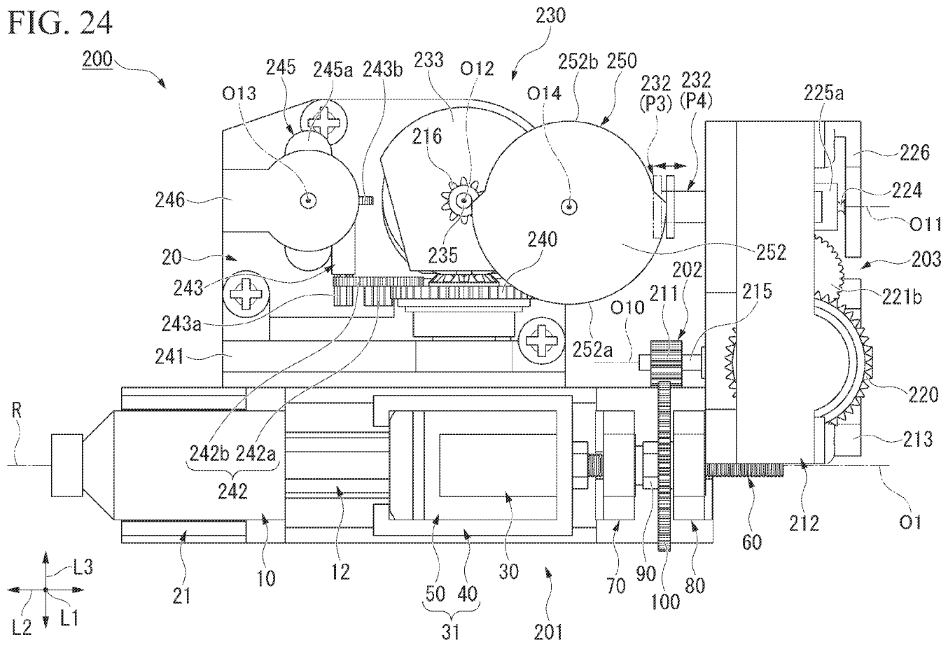

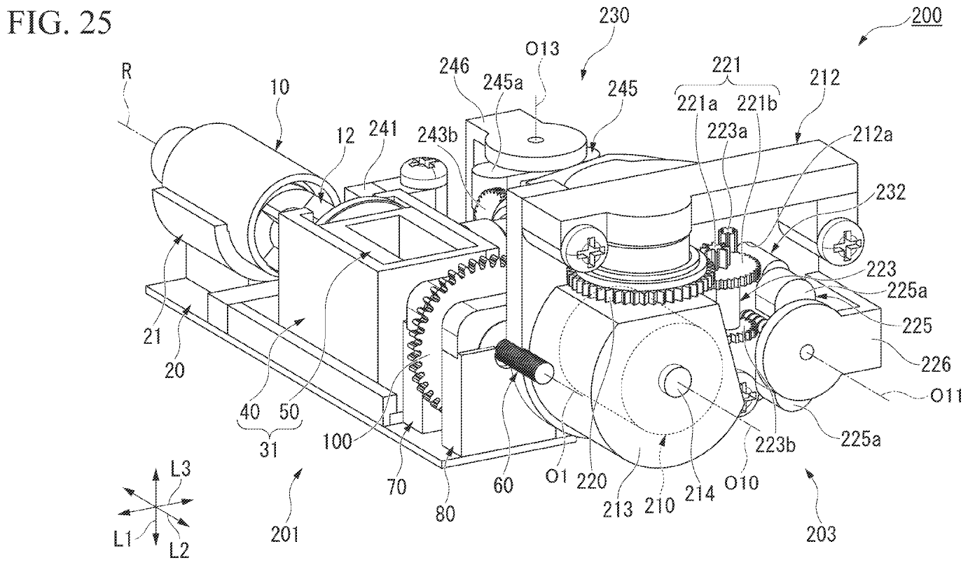

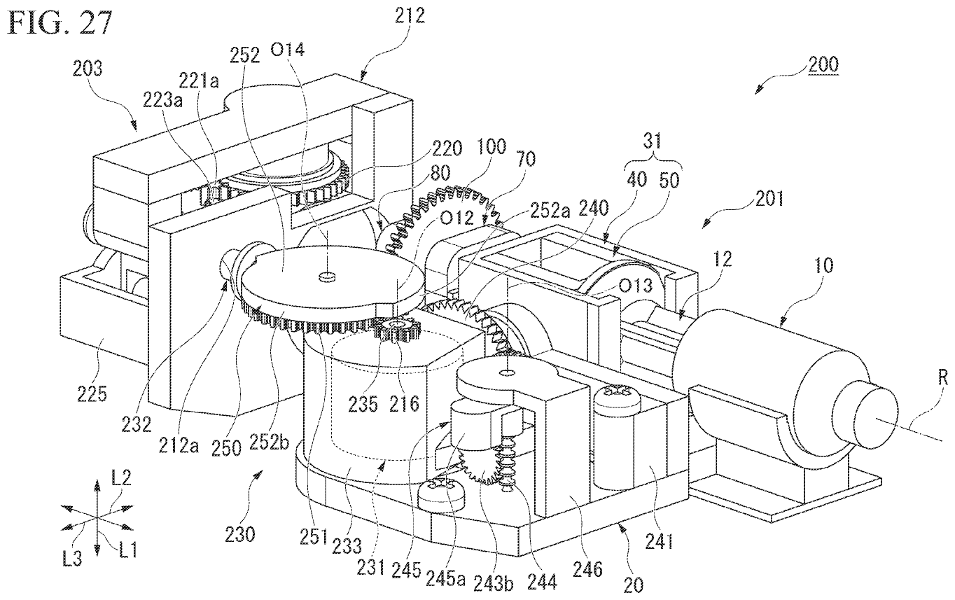

As illustrated in FIGS. 24 to 28, in a chemical solution pump (quantitative amount feed mechanism according to the present invention) 200 of the present embodiment, a feed mechanism 201 includes a mainspring (drive source according to the present invention) 210 that generates the power for rotating the nut member 90, a train wheel mechanism 202 that transmits the power from the mainspring 210 to the nut member 90, and a speed control mechanism 203 that controls the speed of the train wheel mechanism 202.

The train wheel mechanism 202 has a transmission wheel 211 that rotates around a tenth axis O10 by using the power from the mainspring 210 and meshes with the feed wheel 100.

The mainspring 210 is accommodated in a box-shaped accommodation portion 213 fixed to a fixing plate 212 integrally combined with the second support portion 80. As in the mainspring 91 in the first embodiment, the mainspring 210 is equivalent to that used in the mechanical timepiece, is formed in a spiral shape, and can generate the power by the unwinding operation.

An outer end portion of the mainspring 210 is attached to the inside of the accommodation portion 213, and an inner end portion thereof is locked to the drive shaft portion 214 penetrating the accommodation portion 213 in the forward-rearward direction. The drive shaft portion 214 is disposed coaxially with the tenth axis O10. In this manner, the drive shaft portion 214 can rotate around the tenth axis O10 by the unwinding operation of the mainspring 210.

The transmission wheel 211 is disposed on the front side of the accommodation portion 213, and is fixed to a connection shaft portion 215 disposed coaxially with the tenth axis O10. The connection shaft portion 215 is connected to the drive shaft portion 214 inside the accommodation portion 213. In this manner, the transmission wheel 211 can be rotated around the tenth axis O10 via the drive shaft portion 214 and the connection shaft portion 215 by the power generated in association with the unwinding operation of the mainspring 210, and the feed wheel 100 and the nut member 90 can be rotated around the first axis.

In the present embodiment, the nut member 90 is disposed between the first support portion 70 and the feed wheel 100.

A rear end portion side protruding rearward from the accommodation portion 213 in the drive shaft portion 214 is rotated. In this manner, the mainspring 210 can be wound to reduce the diameter. In this case, the connection shaft portion 215 is coaxial with the drive shaft portion 214. Accordingly, the connection shaft portion 215 can also be rotated when wound by the drive shaft portion 214, and the plunger 12 can return in a direction opposite to a driving direction.

The speed control mechanism 203 includes a first intermediate wheel 220 disposed above the accommodation portion 213 and rotating around the axis parallel to the upward-downward direction in association with the rotation of the drive shaft portion 214, a second intermediate wheel 221 rotating in association with the rotation of the first intermediate wheel 220, a third intermediate wheel 223 rotating in association with the rotation of the second intermediate wheel 221, a worm shaft 224 rotating around an eleventh axis O11 parallel to the forward-rearward direction in association with the rotation of the third intermediate wheel 223, and an impeller 225 rotating around the eleventh axis O11 in association with the rotation of the worm shaft 224 and applying rotational resistance to the rotation of the worm shaft 224.

The first intermediate wheel 220 meshes with the drive shaft portion 214 via a bevel wheel (not illustrated). In this manner, the first intermediate wheel 220 can rotate around an axis orthogonal to the tenth axis O10. A clutch mechanism (not illustrated) is provided between the first intermediate wheel 220 and the drive shaft portion 214. In the clutch mechanism, when the drive shaft portion 214 rotates in a winding direction of the mainspring 210, the drive shaft portion 214 is idled with respect to the first intermediate wheel 220. When the drive shaft portion 214 rotates in association with the unwinding operation of the mainspring 210, the drive shaft portion 214 and the first intermediate wheel 220 are rotated together. In this manner, the first intermediate wheel 220 can rotate only when the mainspring 210 is unwound.

The second intermediate wheel 221 includes a second intermediate pinion 221a that meshes with the first intermediate wheel 220, and a second intermediate gear 221b. The third intermediate wheel 223 includes a third intermediate pinion 223a that meshes with the second intermediate gear 221b, and a third intermediate gear 223b that meshes with the worm shaft 224. The second intermediate wheel 221 and the third intermediate wheel 223 are supported by the fixing plate 212 via a connection piece (not illustrated).

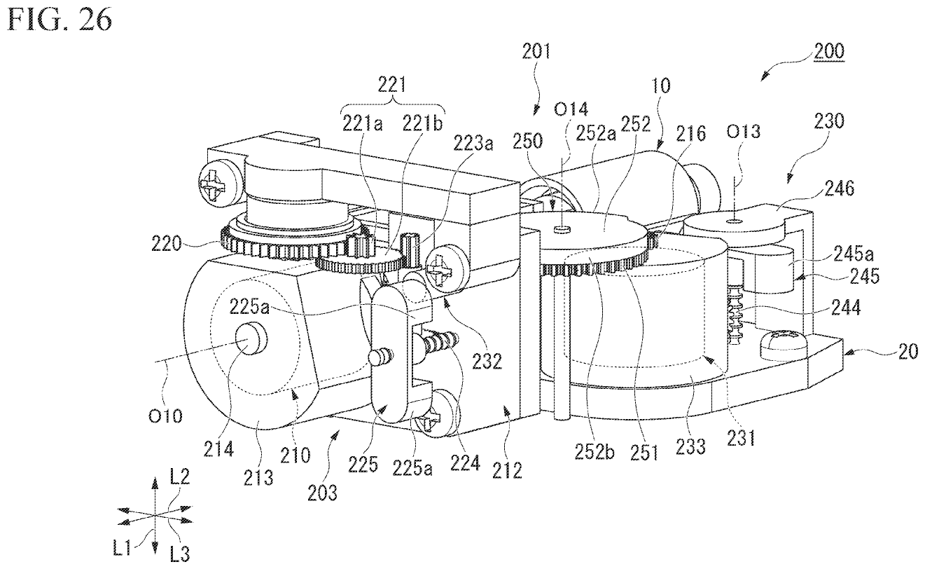

The front end portion of the worm shaft 224 is pivotally supported to be rotatable by the fixing plate 212, and the rear end portion is pivotally supported to be rotatable by a connection piece 226 attached to the fixing plate 212. A spiral worm groove is formed over the entire length on the outer peripheral surface of the worm shaft 224. The above-described third intermediate gear 223b meshes with the worm groove. In FIG. 26, the connection piece 226 is omitted in the illustration.

The impeller 225 is integrally combined with the worm shaft 224, and includes a pair of blade plates 225a. Therefore, the impeller 225 rotates while receiving air resistance generated by the blade plate 225a in association with the rotation of the worm shaft 224. In this manner, the impeller 225 can apply rotational resistance to the rotation of the worm shaft 224. The number of blade plates 225a is not limited to one pair. The number may be one, or may be three or more.

In particular, the impeller 225 receives the air resistance corresponding to the rotation speed of the worm shaft 224 via the blade plate 225a. Accordingly, it is possible to control the speed of the whole train wheel mechanism 202.

Furthermore, the feed mechanism 201 of the present embodiment includes a switching mechanism 230 that switches between stopping and starting the power transmission from the mainspring 210 to the nut member 90.

The switching mechanism 230 includes a mainspring (switching mainspring according to the present invention) 231 that generates switching power by the unwinding operation, and a movable pin (movable member according to the present invention) 232 that moves between a separation position P3 (refer to FIG. 24) separated from the impeller 225 by the switching power and a stop position P4 (refer to FIG. 24) which comes into contact with the impeller 225 to stop the rotation of the impeller 225.

The mainspring 231 is accommodated inside a box-shaped accommodation portion 233 fixed onto the base plate 20. As in the mainspring 91 in the first embodiment, the mainspring 231 is equivalent to that used in the mechanical timepiece, is formed in a spiral shape, and can generate the switching power by the unwinding operation.

The outer end portion of the mainspring 231 is attached the inside of the accommodation portion 233, and the inner end portion is locked to a drive shaft portion (not illustrated) penetrating the accommodation portion 233 in the upward-downward direction. The drive shaft portion is disposed coaxially with a twelfth axis line O12. In this manner, the drive shaft portion can be rotated around the twelfth axis O12 by the unwinding operation of the mainspring 231.

In the base plate 20, an opening portion (not illustrated) that accommodates a lower end portion of the drive shaft portion is formed in a portion located below the accommodation portion 233. Then, the lower end portion of the drive shaft portion located inside the opening portion so that the mainspring 231 can be wound to reduce the diameter.

In the drive shaft portion, a connection shaft portion 235 protruding upward from the accommodation portion 233 is disposed coaxially with the twelfth axis O12. The connection shaft portion 235 is connected to the drive shaft portion inside the accommodation portion 233. In this manner, the drive shaft portion and the connection shaft portion 235 can be rotated around the twelfth axis O12 by the power generated in association with the unwinding operation of the mainspring 231. A connection gear 216 is fixed to the connection shaft portion 235.

A first intermediate wheel 240 rotating around the axis parallel to the rightward-leftward direction in association with the rotation of the drive shaft portion is disposed between the accommodation portion 233 and the movable case 31. The first intermediate wheel 240 is pivotally supported by the connection piece 241 fixed to the base plate 20.

The first intermediate wheel 240 meshes with the drive shaft portion via a bevel wheel (not illustrated). In this manner, the first intermediate wheel 240 can rotate around the axis orthogonal to the twelfth axis O12. A clutch mechanism (not illustrated) is provided between the first intermediate wheel 240 and the drive shaft portion. In the clutch mechanism, when the drive shaft portion is rotated in a winding direction of the mainspring 231, the drive shaft portion is idled with respect to the first intermediate wheel 240. When the drive shaft portion is rotated in association with an unwinding operation of the mainspring 231, the drive shaft portion and the first intermediate wheel 240 are rotated together. In this manner, the first intermediate wheel 240 can rotate only when the mainspring 231 is unwound.

In addition, the base plate 20 has a second intermediate wheel 242 rotating in association with the rotation of the first intermediate wheel 240, a third intermediate wheel 243 rotating in association with the rotation of the second intermediate wheel 242, a worm shaft 244 rotating around a thirteenth axis O13 parallel to the upward-downward direction in association with the rotation of the third intermediate wheel 223, and an impeller 245 rotating around the thirteenth axis O13 in association with the rotation of the worm shaft 244 and applying the rotational resistance to the rotation of the worm shaft 244.

The second intermediate wheel 242 includes a second intermediate pinion 242a that meshes with the first intermediate wheel 240, and a second intermediate gear 242b. The third intermediate wheel 243 includes a third intermediate pinion 243a that mesh with the second intermediate gear 242b, and a third intermediate gear 243b that meshes with the worm shaft 244. The second intermediate wheel 242 and the third intermediate wheel 243 are supported on the base plate 20 via a connection piece (not illustrated).

The lower end portion of the worm shaft 244 is pivotally supported to be rotatable by the base plate 20, and the upper end portion is pivotally supported to be rotatable by the connection piece 246 attached to the base plate 20. A spiral worm groove is formed over the entire length on the outer peripheral surface of the worm shaft 244. The above-described third intermediate gear 243b meshes with the worm groove.

The impeller 245 is integrally combined with the worm shaft 244, and includes a pair of blade plates 245a. Therefore, the impeller 245 rotates while receiving air resistance from the blade plate 245a in association with the rotation of the worm shaft 244. In this manner, the impeller 245 can apply the rotational resistance to the rotation of the worm shaft 244. The number of blade plates 245a is not limited to one pair. The number may be one, or may be three or more.

In particular, the impeller 245 receives the air resistance corresponding to the rotation speed of the worm shaft 244 via the blade plate 245a. Accordingly, the speed of the connection gear 216 can be controlled.

A cam gear 250 pivotally supported by the base plate 20 meshes with the connection gear 216. The cam gear 250 includes a connection target gear 251 rotatable around a fourteenth axis O14 parallel to the upward-downward direction and meshing with the connection gear 216, and a cam plate 252 integrally formed with the connection target gear 251.

Therefore, the cam gear 250 is rotated by the power generated in association with the unwinding operation of the mainspring 231, and can be rotated in association with the rotation of the connection gear 216 whose rotation speed is controlled by the impeller 245.

The cam plate 252 is formed in a disc shape in which two outer peripheral portions having different outer diameters, that is, a first outer peripheral portion 252a and a second outer peripheral portion 252b are connected to each other in the circumferential direction. In the illustrated example, the outer diameter of the first outer peripheral portion 252a is formed to be larger than that of the second outer peripheral portion 252b. The length of the first outer peripheral portion 252a in the circumferential direction and the length of the second outer peripheral portion 252b in the circumferential direction are equal to each other.

However, without being limited to this case, a ratio of the length of the first outer peripheral portion 252a in the circumferential direction to the length of the second outer peripheral portion 252b in the circumferential direction may be changed as appropriate.

The movable pin 232 is inserted into a through-hole 212a penetrating the fixing plate 212 in the forward-rearward direction to be slidably in the forward-rearward direction L2. In this case, the movable pin 232 is disposed so that the front end portion is located behind the cam plate 252 and the rear end portion is located in front of the blade plate 225a in the impeller 225. The front end surface and the rear end surface of the movable pin 232 are both flat surfaces.

The movable pin 232 is always biased rearward (to the cam plate 252 side) by a biasing member such as a coil spring (not illustrated). Therefore, the front end portion of the movable pin 232 comes into contact with either the first outer peripheral portion 252a or the second outer peripheral portion 252b in the cam plate 252 from the rear.

When the front end portion of the movable pin 232 is in contact with the first outer peripheral portion 252a having a large outer diameter, the rear end portion comes into contact with the impeller 225 from the front. When the front end portion is in contact with the second outer peripheral portion 252b having a small outer diameter, the rear end portion is separated from the impeller 225.

Therefore, a position where the front end portion of the movable pin 232 comes into contact with the first outer peripheral portion 252a is the above-described stop position P4, and a position where the front end portion of the movable pin 232 comes into contact with the second outer peripheral portion 252b is the above-described separation position P3.

### Operation of Chemical Solution Pump

According to the chemical solution pump 200 of the present embodiment configured as described above can also achieve operational effects the same as those of the first embodiment.

In addition, in a case of the present embodiment, both the speed control mechanism 203 and the switching mechanism 230 include the mainsprings 210 and 231. Accordingly, only a wind noise is generated as in the second embodiment, and it is possible to provide the chemical solution pump 200 which is excellently quiet.

Furthermore, the switching mechanism 230 is provided. Accordingly, it is possible to adjust a discharge timing or a discharge time of the chemical solution W from the syringe 10. Accordingly, it is possible to provide the chemical solution pump 200 which achieves convenient use and can be used in various ways corresponding to various purposes. Moreover, the switching mechanism 230 is operated by using the power of the mainspring 231. Accordingly, electric power is not needed. Therefore, the discharge timing of the chemical solution W can be controlled without using the electric power, and it is possible to provide the chemical solution pump 200 which achieves extremely convenient use.

This configuration will be described in detail.

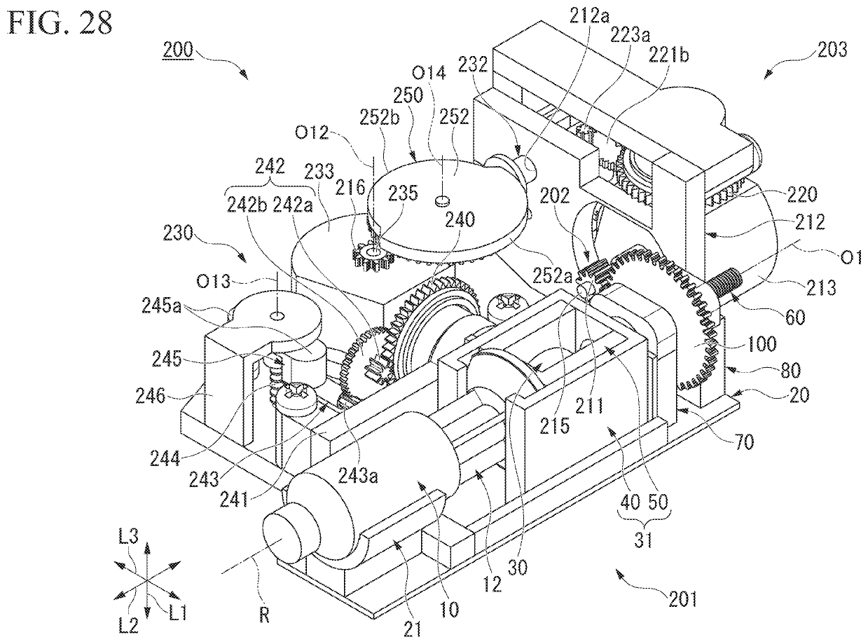

As illustrated in FIG. 28, when the front end portion of the movable pin 232 is in contact with the first outer peripheral portion 252a in the cam plate 252, the movable pin 232 is located at the stop position P4. Accordingly, the rear end portion of the movable pin 232 is in contact with the impeller 225. Therefore, the impeller 225 can be stopped, and in association therewith, the whole train wheel mechanism 202 (first intermediate wheel 220, second intermediate wheel 221, and third intermediate wheel 223) can be stopped. Therefore, the transmission wheel 211 (train wheel mechanism 202) can be stopped, and the feed wheel 100 and the nut member 90 can be maintained in a stopped state. Therefore, the movement of the plunger 12 can be stopped, and the operation for discharging the chemical solution W can be maintained in a stopped state.

On the other hand, the connection gear 216 in the switching mechanism 230 rotates around the twelfth axis O12 by the power generated in association with the unwinding operation of the mainspring 231. In this case, the speed of the connection gear 216 is controlled by the rotation of the impeller 245, and the connection gear 216 rotates at a constant rotation speed. Therefore, the cam gear 250 rotates around the twelfth axis O12 at a constant rotation speed in association with the rotation of the connection gear 216.

Then, when a portion in contact with the front end portion of the movable pin 232 is switched from the first outer peripheral portion 252a to the second outer peripheral portion 252b by the rotation of the cam gear 250, the second outer peripheral portion 252b has the outer diameter smaller than that of the first outer peripheral portion 252a. Accordingly, the movable pin 232 moves forward due a biasing force of a biasing member. In this manner, the movable pin 232 is shifted to the separation position P3 at which the front end portion comes into contact with the second outer peripheral portion 252b and the rear end portion is separated from the impeller 225.

In this manner, the impeller 225 of the speed control mechanism 203 can be rotated. While the speed of the train wheel mechanism 202 is controlled, the power generated in association with the unwinding operation of the mainspring 210 can be transmitted to the feed wheel 100 and the nut member 90 via the transmission wheel 211. Accordingly, it is possible to move the plunger 12 with a constant movement amount, and the chemical solution W can be discharged.

Hitherto, the embodiments of the present invention have been described. However, the embodiments are presented as examples, and are not intended to limit the scope of the invention. The embodiment can be implemented in various other forms. Various omissions, substitutions, and modifications can be made within the scope not departing from the concept of the invention. For example, the embodiments and modification examples thereof include those which can be easily assumed by those skilled in the art, and include those which are substantially the same, those which have an equivalent range.

For example, in the respective above-described embodiments, the chemical solution administration device in which the indwelling needle is provided in the main body case accommodating the chemical solution pump has been described as an example. However, the present invention is not limited to this case. For example, the chemical solution administration device may be provided as follows. A patch portion including the indwelling needle may be provided separately from the main body case, and the main body case and the patch portion may be connected to each other by a flexible tube.

Furthermore, in the above-described respective embodiments, a configuration is adopted so that the feed screw is connected in series to the plunger via the movable case. However, the present invention is not limited to this case. For example, a configuration may be adopted so that the movable case and the feed screw are connected in parallel to the plunger 12. Even in this case, the same operational effects can be achieved.

In addition, in the above-described respective embodiments, a so-called lead screw method is adopted in which the feed screw is moved forward by the rotation of the nut member. However, the present invention is not limited to this case.

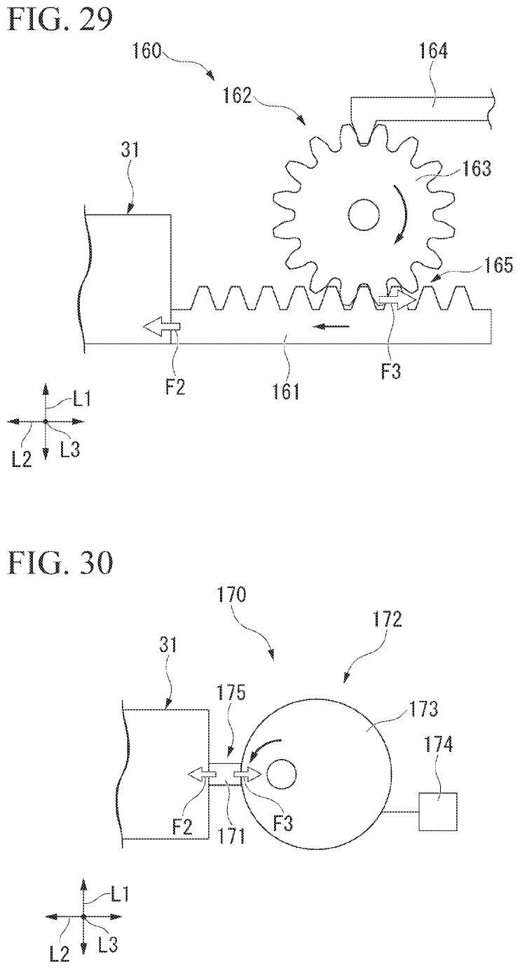

For example, as illustrated in FIG. 29, an adjustment mechanism 160 may be provided which includes a rack portion (movable body according to the present invention) 161 disposed on the rear side of the movable case 31 and moving in conjunction with the plunger 12 via the movable case 31, and a feed mechanism 162 moving the rack portion 161 forward at a predetermined speed and applying the auxiliary force F2 to the plunger 12 via the rack portion 161.

The feed mechanism 162 includes a pinion 163 that meshes with a rack teeth in the rack portion 161 and a speed control lever 164 that controls the rotation speed of the pinion 163.

In this case, the adjustment mechanism 160 can be configured to adopt a so-called rack and pinion method. Even in this case, the same operational effects can be achieved. In this case, a meshing force between the pinion 163 and the rack teeth can be used as the reaction force F3. Therefore, the pinion 163 and the rack portion 161 can function as a braking unit 165.

Furthermore, as illustrated in FIG. 30, an adjustment mechanism 170 may be provided which includes a movable rod (movable body according to the present invention) 171 disposed on the rear side of the movable case 31 and moving in conjunction with the plunger 12 via the movable case 31, and a feed mechanism 172 moving the movable rod 171 forward at a predetermined speed and applying the auxiliary force F2 to the plunger 12 via the movable rod 171.

The feed mechanism 172 includes an eccentric cam 173 that presses the movable rod 171 by rotating around the rotation axis, and a speed control unit 174 that rotates the eccentric cam 173 and controls the rotation speed.

In this case, the adjustment mechanism 170 can be configured to adopt a so-called cam method, and the same effect can be achieved. In this case, the frictional force between the movable rod 171 and the peripheral surface of the eccentric cam 173 can be used as the reaction force F3. Therefore, the movable rod 171 and the eccentric cam 173 can function as the braking unit 175.

In addition, instead of the eccentric cam 173, for example, an inclined cam inclined with respect to the rotation axis may be used to press the movable rod 171 by the rotation of the inclined cam, or the movable rod may be pressed by the inclined lever inclined about the rotation axis.

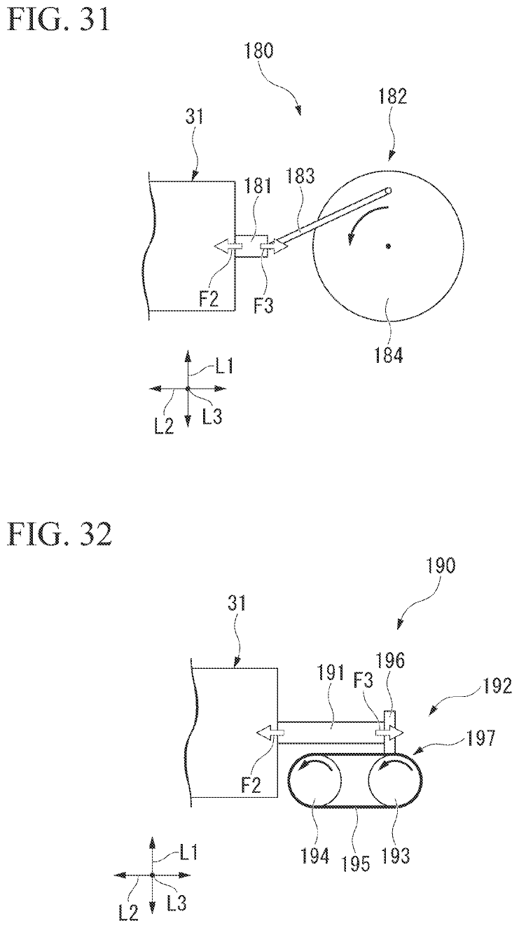

In addition, as illustrated in FIG. 31, an adjustment mechanism 180 may be provided which includes a movable rod (movable body according to the present invention) 181 that is disposed on the rear side of the movable case 31 and moves in conjunction with the plunger 12 via the movable case 31, and a feed mechanism 182 moving the movable rod 181 forward at a predetermined speed and applying the auxiliary force F2 to the plunger 12 via the movable rod 181.

The feed mechanism 182 is connected to the movable rod 181 via a link rod 183, and includes a rotating plate 184 that rotates around the rotation axis.

In this case, the adjustment mechanism 180 can be configured by a so-called gear system, and the same effect can be achieved. In this case, for example, the rotational resistance of the rotating plate 184 can be used as the reaction force F3.

In addition, as illustrated in FIG. 32, an adjustment mechanism 190 may be provided which includes a movable rod (movable body according to the present invention) 191 that is disposed on the rear side of the movable case 31 and move in conjunction with the plunger 12 via the movable case 31, and a feed mechanism 192 moving the movable rod 191 forward at a predetermined speed and applying the auxiliary force F2 to the plunger 12 via the movable rod 191.

The feed mechanism 192 includes a drive belt 195 wound around the first roller 193 and the second roller 194 and moved at a predetermined speed by the rotation of both rollers 193 and 194, and a moving piece 196 provided in the drive belt 195 and moving the movable rod 191 in association with the movement of the drive belt 195.

In this case, the adjustment mechanism 190 can be configured by a so-called belt drive system, and the same operational effects can be achieved. In this case, a frictional force between a first roller 193 and a second roller 194 and a drive belt 195 can be used as the reaction force F3. Therefore, the first roller 193, the second roller 194, and the drive belt 195 can function as a braking unit 197.

As described above, examples of the adjustment mechanism for adjusting the movement of the plunger have been described with reference to FIGS. 29 to 32. However, the present invention is not limited to these cases, and may be changed as appropriate.

Furthermore, in the above-described respective embodiments, a case where the plunger is pressed by using the elastic restoring force of the spiral spring as the driving force has been described as an example. However, the case is not limited to the spiral spring. For example, the elastic restoring force of various spring members other than spiral springs such as a leaf spring, a coil spring, a torsion springs, a disc springs, and a volute spring may be used as the driving force.

Furthermore, without being limited to the spring members, the driving force may be generated by using a compressed fluid such as a compressed gas or a compressed liquid. The driving force may be generated by using the stretching and shrinking property of the shape memory alloy wire. Alternatively, the driving force may be generated by using a repulsive force based on a magnetic force.

Furthermore, in the above-described second embodiment, the oscillation of the oscillator plate which is generated by the stretching and shrinking property of the first shape memory alloy wire and the second shape memory alloy wire is used to switch between stopping and starting the power transmission from the mainspring to the nut member. However, the present invention is not limited to this case. For example, a configuration may be adopted so that the power starts to be transmitted by using a meshing operation of gears.

For example, in an intermediate portion of a transmission route for transmitting the power from the mainspring to the nut member, there may be provided a switching mechanism including an oscillation wheel rotating based on the power transmitted from the mainspring, a driven wheel that transmits the power from the oscillation wheel side to the nut member side when the oscillation wheel meshes with the driven wheel, and an oscillation lever that meshes with the driven wheel by causing the oscillation wheel to oscillate after a lapse of a predetermined time.

In this case, for example, even when the drive wheel starts to be rotated by the power of the mainspring, in a stage before the lapse of the predetermined time, it is possible to prevent the power from being transmitted to the nut member. Then, when the predetermined time elapses and it is a timing required for chemical solution administration, the oscillation lever causes the oscillation wheel to oscillate to mesh with the driven wheel. In this manner, the power from the mainspring can be transmitted to the nut member via the oscillation wheel and the driven wheel, and the chemical solution administration can start.

Therefore, operational effects the same as those of the second embodiment can be achieved. In particular, in a case of this configuration, unlike the second embodiment, the electric power for energizing the shape memory alloy wire is not needed. Therefore, the timing for administering the chemical solution can be controlled without using the electric power.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A chemical solution pump comprising:
a holding member that holds a syringe filled with a chemical solution and discharging the chemical solution in association with a movement of a plunger disposed to be slidably movable inside the syringe;
a drive unit that presses the plunger with a predetermined driving force to move the plunger in a first direction toward an inside of the syringe; and
an adjustment mechanism that adjusts the movement of the plunger so that the plunger moves with the driving force,
wherein the adjustment mechanism applies an auxiliary force to the plunger in a direction the same as the first direction, or applies a reaction force to the plunger in a second direction opposite to the first direction, in accordance with a difference between a resistance force generated by the movement of the plunger inside the syringe and the driving force.

2. The chemical solution pump according to claim 1,
wherein the adjustment mechanism includes
a movable body disposed to be movable along an axis in conjunction with the plunger,
a feed mechanism that moves the movable body in the first direction at a predetermined speed, and applies the auxiliary force to the plunger via the movable body, and
a braking unit that applies the reaction force to the plunger in the second direction via the movable body.

3. The chemical solution pump according to claim 2,
wherein the movable body has a feed screw having a male screw portion formed on an outer peripheral surface, and disposed in a state where rotation around the axis is restricted,
the feed mechanism includes
a nut member that has a female screw portion screwed to the male screw portion, and is screwed to the feed screw,
a drive source that generates power for rotating the nut member,
a train wheel mechanism that transmits the power from the drive source to the nut member, and
a speed control mechanism that controls a speed of the train wheel mechanism, and
the braking unit uses at least a meshing force in the train wheel mechanism and a meshing force of the male screw portion with respect to the female screw portion, as the reaction force.

4. The chemical solution pump according to claim 3,
wherein the drive source has a mainspring that generates the power by an unwinding operation.

5. The chemical solution pump according to claim 4,
wherein the feed mechanism includes a switching mechanism that switches between stopping and starting power transmission from the drive source to the nut member, and
the feed mechanism stops the movement of the feed screw and the plunger by stopping the power transmission to the nut member, and moves the plunger, based on the driving force while causing the adjustment mechanism to adjust the movement of the plunger by starting the power transmission to the nut member.

6. The chemical solution pump according to claim 5,
wherein the speed control mechanism includes an impeller that meshes with the train wheel mechanism, and is rotated by the power associated with the unwinding operation of the mainspring,
the impeller generates resistance corresponding to a rotation speed of the train wheel mechanism to control the speed of the train wheel mechanism, and
the switching mechanism includes
a switching mainspring that generates switching power by an unwinding operation, and
a movable member that moves between a separation position separated from the impeller and a stop position in contact with the impeller to stop rotation of the impeller, based on the switching power.

7. The chemical solution pump according to claim 1,
wherein the drive unit includes a spring member that generates the driving force by using an elastic restoring force.

8. A chemical solution administration device comprising:
the chemical solution pump according to claim 1;
a main body case internally accommodating the chemical solution pump, and mountable on a living body surface; and
an indwelling needle capable of indwelling the living body surface in a state where a living body is punctured, and into which the chemical solution discharged from the syringe is introduced.

9. A chemical solution administration device comprising:
the chemical solution pump according to claim 2;
a main body case internally accommodating the chemical solution pump, and mountable on a living body surface; and
an indwelling needle capable of indwelling the living body surface in a state where a living body is punctured, and into which the chemical solution discharged from the syringe is introduced.

10. A chemical solution administration device comprising:
the chemical solution pump according to claim 3;
a main body case internally accommodating the chemical solution pump, and mountable on a living body surface; and
an indwelling needle capable of indwelling the living body surface in a state where a living body is punctured, and into which the chemical solution discharged from the syringe is introduced.

11. A chemical solution administration device comprising:
the chemical solution pump according to claim 4;
a main body case internally accommodating the chemical solution pump, and mountable on a living body surface; and
an indwelling needle capable of indwelling the living body surface in a state where a living body is punctured, and into which the chemical solution discharged from the syringe is introduced.

12. A chemical solution administration device comprising:
the chemical solution pump according to claim 5;
a main body case internally accommodating the chemical solution pump, and mountable on a living body surface; and
an indwelling needle capable of indwelling the living body surface in a state where a living body is punctured, and into which the chemical solution discharged from the syringe is introduced.

13. A chemical solution administration device comprising:
the chemical solution pump according to claim 6;
a main body case internally accommodating the chemical solution pump, and mountable on a living body surface; and
an indwelling needle capable of indwelling the living body surface in a state where a living body is punctured, and into which the chemical solution discharged from the syringe is introduced.

14. A chemical solution administration device comprising:
the chemical solution pump according to claim 7;
a main body case internally accommodating the chemical solution pump, and mountable on a living body surface; and
an indwelling needle capable of indwelling the living body surface in a state where a living body is punctured, and into which the chemical solution discharged from the syringe is introduced.
